(19) United States
(12) Patent Application Publication
(10) Pub. No.: US 2026/0306590 A1
(43) Pub. Date: Oct. 8, 2026

(54) TREATING MUSCLE WEAKNESS WITH ALKALINE PHOSPHATASES

(71) Applicant: ALEXION PHARMACEUTICALS, INC., Boston, MA (US)

(72) Inventors: Denise DEVORE, Boston, MA (US); Anna PETRYK, Boston, MA (US); Walter VOEGTLI, Boston, MA (US); Derek DUNN, Boston, MA (US); Juan RUANOVA, Boston, MA (US)

(21) Appl. No.: 19/480,505

(22) PCT Filed: Mar. 26, 2024
(86) PCT No.: PCT/US2024/021544

§ 371 Date: Oct. 31, 2025

Related U.S. Application Data
(60) Provisional application No. 63/463,539, filed on May 2, 2023.

(51) Int. Cl. A61K 38/46; A61P 21/00

(52) U.S. Cl. CPC A61K 38/465; A61P 21/00; C12Y 301/03001

(57) ABSTRACT

The disclosure features methods for treating or ameliorating at least one symptom of a subject having or being prone to a muscle weakness disease by administering to the subject a therapeutically effective amount of at least one recombinant polypeptide having alkaline phosphatase activity. The subject is characterized as lacking a loss-of-function mutation in an ALPL gene.

### CROSS REFERENCE TO RELATED APPLICATIONS

This application claims priority to, and the benefit of, U.S. Provisional Application No. 63/463,539, filed May 2, 2023, the entire contents which are incorporated herein by reference.

### SEQUENCE LISTING

The instant application contains a Sequence Listing which has been submitted electronically in XML file format and is hereby incorporated by reference in its entirety. Said XML copy, created on Mar. 22, 2024, is named 0467WO_SL.xml and is 31,813 bytes in size.

### BACKGROUND

Hypophosphatasia (HPP)-like diseases are caused by unknown etiologies and result in a remarkable range of symptoms and severity, from rickets (osteomalacia) to almost complete absence of bone mineralization in utero. Most subjects exhibit characteristics of skeletal changes, short stature, painful lower limbs, gait disturbance, and premature shedding of teeth. Furthermore, these subjects exhibit muscle weakness and hypotonia, severely lowering their quality of life due to the physical impairments that these types of diseases impart on a daily basis.

### SUMMARY

Featured is a method of treating or reducing the risk of developing muscle weakness in a subject having or at risk of having a muscle weakness disease by administering to the subject a therapeutically effective amount of at least one recombinant polypeptide having alkaline phosphatase activity, wherein the subject exhibits a reduced concentration of tissue non-specific alkaline phosphatase (TNSALP), and wherein the subject does not have a loss-of-function mutation in an ALPL gene.

The reduced concentration of TNSALP may be caused by reduced transcription of ALPL, reduced translation of ALPL mRNA, elevated or reduced posttranslational modification of TNSALP, or reduced of TNSALP enzymatic activity. The reduced transcription of ALPL, reduced translation of ALPL mRNA, elevated or reduced posttranslational modification of TNSALP, or reduced TNSALP enzymatic activity may be measured relative to a normal subject.

The subject may have a mutation in a 3′ untranslated region (UTR), a 5′ UTR, or intronic region of the ALPL gene. The subject may have a mutation in one or more of ATP1A3, ANKH, ENPP1, FGFR3, PHOSPHO1, PTHIR, PTH2R, SPP1, TNFRSF11A, TNFRSF11B, COL1A1, COL1A2, SOX9, PDXP, AOX1, PNPO, PDXK, ADCK3, MTRNR2, and S1PR1. The subject may have an ATP1A3 variant c.357+1G>A. The subject may have an ADCK3 variant m. 1665G>A. The subject may have an MTRNR2 variant m. 1836A>G.

A muscle (e.g., a leg muscle, such as a soleus muscle or an extensor digitorum longus (EDL) muscle) of said subject is not significantly different from a muscle of a normal subject without said muscle weakness disease in at least one property selected from muscle fiber type proportion and fiber contractile properties.

The muscle weakness disease may be caused by reduced alkaline phosphatase activity. The subject may have an elevated serum concentration of pyrophosphate (PPi), and/or the muscle weakness disease may be caused by the elevated concentration of PPi. The elevated concentration of pyrophosphate (PPi) may enhance muscle weakness in said subject. Administration of the recombinant polypeptide reduces the concentration of PPi in the subject (e.g., by 10%, 20%, 30%, 40%, 50%, 60%, 70%, 80%, 90%, or 100%) relative to an untreated subject.

The recombinant polypeptide may be administered to the subject daily for at least one week, one month, three months, six months, or one year. The recombinant polypeptide may be administered subcutaneously, intravenously, intramuscularly, sublingually, intrathecally, or intradermally.

The recombinant polypeptide may include at least one of a tissue nonspecific alkaline phosphatase (TNALP), a placental alkaline phosphatase (PALP), a germ cell alkaline phosphatase (GCALP), an intestinal alkaline phosphatase (IALP), and biologically functional fragments, fusions, or chimeric constructs thereof. The recombinant polypeptide may include at least one of a soluble fragment of TNALP, PALP, GCALP, and IALP. The tissue nonspecific alkaline phosphatase (TNALP) may contain amino acids 1-485 of SEQ ID NO: 1.

The recombinant polypeptide may be a fusion protein. The recombinant polypeptide may include an immunoglobulin molecule, such as a fragment crystallizable (Fc) region. The Fc may have an amino acid sequence of SEQ ID NO: 20. The recombinant polypeptide may include a negatively charged peptide. The negatively charged polypeptide may include one to fifty negatively charged amino acids, such as an aspartic acid or glutamic acid. The negatively charged peptide may include at least one of D₁₀, D₁₆, E₁₀, and E₁₆.

The recombinant polypeptide may include a bone targeted alkaline phosphatase having the structure:

Z-sALP-Y-spacer-X-Wₙ-V,

wherein sALP is the extracellular domain of the alkaline phosphatase;
V is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid; and
Wₙ is a polyaspartate or a polyglutamate wherein n=10 to 16.
   The spacer may include an Fc region (e.g., an FC having an amino acid sequence of SEQ ID NO: 20). The recombinant polypeptide may include the structure of sALP-Fc-D₁₀.

The recombinant polypeptide may be administered at a dosage of from about 0.1 mg/kg/day to about 20 mg/kg/day (e.g., about 0.5 mg/kg/day to about 20 mg/kg/day, about 0.5 mg/kg/day to about 10 mg/kg/day, about 1 mg/kg/day to about 10 mg/kg/day, e.g., about 6 mg/kg/week), or a comparable weekly dosage. The recombinant polypeptide may be administered at a dosage of about 1 mg/kg 6 times per week, about 2 mg/kg 3 times per week, or 3 mg/kg 2 times per week.

Prior to administration of the recombinant polypeptide, the subject may be characterized as having an average walking distance in six minutes of about 350 meters or less. Administration of the recombinant polypeptide may promote an increase in an average walking distance in six minutes by the subject of at least 100 meters or more. The subject may exhibit an average walking distance in six minutes of about 500 meters or more after administration of the recombinant polypeptide. The subject may exhibit decreased reliance on an assistive mobility device (e.g., a walker, a wheelchair, braces, crutches, and orthotics) after administration of the recombinant polypeptide.

Prior to administration of the recombinant polypeptide, the subject may be characterized as having a plasma PPi concentration of about 4.5 μM or greater. Administration of the recombinant polypeptide may promote a median decrease in PPi concentration in a plasma sample from the subject of at least about 1 μM. The subject may exhibit a plasma PPi concentration of about 2 μM to about 5 UM after administration of the recombinant polypeptide.

In some embodiments,
i) the subject is 0 to 14 days of age and, prior to administration of the recombinant polypeptide, is characterized as having a plasma ALP concentration of about 90 U/L or less;
ii) the subject is 15 days to less than 1 year of age and, prior to administration of the recombinant polypeptide, is characterized as having a plasma ALP concentration of about 134 U/L or less;
iii) the subject is about 1 year to less than 10 years of age and, prior to administration of the recombinant polypeptide, is characterized as having a plasma ALP concentration of about 156 U/L or less;
iv) the subject is about 10 years to about 13 years of age and, prior to administration of the recombinant polypeptide, is characterized as having a plasma ALP concentration of about 141 U/L or less;
v) the subject is female and about 13 years to about 15 years of age and, prior to administration of the recombinant polypeptide, is characterized as having a plasma ALP concentration of about 62 U/L or less;
vi) the subject is male and about 13 years to about 15 years of age and, prior to administration of the recombinant polypeptide, is characterized as having a plasma ALP concentration of about 127 U/L or less;
vii) the subject is female and about 15 years to about 17 years of age and, prior to administration of the recombinant polypeptide, is characterized as having a plasma ALP concentration of about 54 U/L or less;
viii) the subject is male and about 15 years to about 17 years of age and, prior to administration of the recombinant polypeptide, is characterized as having a plasma ALP concentration of about 89 U/L or less;
ix) the subject is about 17 years of age or older and, prior to administration of the recombinant polypeptide, is characterized as having a plasma ALP concentration of about 48 U/L or less; or
x) the subject is about 17 years of age or older and, prior to administration of the recombinant polypeptide, is characterized as having a plasma ALP concentration of about 59 U/L or less.
Administration of the recombinant polypeptide may promote a median increase in ALP concentration in a plasma sample from the subject of at least about 100 U/L or greater.

In some embodiments,
i) the subject is 0 to 14 days of age and, after administration of the recombinant polypeptide, is characterized as having a plasma ALP concentration of about 273 U/L or greater;
ii) the subject is 15 days to less than 1 year of age and, after administration of the recombinant polypeptide, is characterized as having a plasma ALP concentration of about 518 U/L or greater;
iii) the subject is about 1 year to less than about 10 years of age and, after administration of the recombinant polypeptide, is characterized as having a plasma ALP concentration of about 369 U/L or greater;
iv) the subject is about 10 years to about 13 years of age and, after administration of the recombinant polypeptide, is characterized as having a plasma ALP concentration of about 460 U/L or greater;
v) the subject is female and about 13 years to about 15 years of age and, after administration of the recombinant polypeptide, is characterized as having a plasma ALP concentration of about 280 U/L or greater;
vi) the subject is male and about 13 years to about 15 years of age and, after administration of the recombinant polypeptide, is characterized as having a plasma ALP concentration of about 517 U/L or greater;
vii) the subject is female and about 15 years to about 17 years of age and, after administration of the recombinant polypeptide, is characterized as having a plasma ALP concentration of about 128 U/L or greater;
viii) the subject is male and about 15 years to about 17 years of age and, after administration of the recombinant polypeptide, is characterized as having a plasma ALP concentration of about 365 U/L or greater;
ix) the subject is female and about 17 year MTRNR2 s of age or older and, after administration of the recombinant polypeptide, is characterized as having a plasma ALP concentration of about 95 U/L or greater; or
x) the subject is male and about 17 years of age or older and, after administration of the recombinant polypeptide, is characterized as having a plasma ALP concentration of about 164 U/L or greater.

Prior to administration of the recombinant polypeptide, the subject may be characterized as having an average Bruininks-Oseretsky Test of Motor Proficiency 2nd Edition (BOT-2) strength score of about 10 or less. Prior to administration of the recombinant polypeptide, the subject may be characterized as having an average BOT-2 running speed and agility score of about 5 or less. Administration of the recombinant polypeptide may result in an average BOT-2 strength score of the subject of about 10 or more. Administration of the recombinant polypeptide may result in an average BOT-2 running speed and agility score of the subject of about 5 or more.

Prior to administration of the recombinant polypeptide, the subject may be characterized as having an average Childhood Health Assessment Questionnaire (CHAQ) index score of about 0.8 or more. Administration of the recombinant polypeptide may result in an average CHAQ index score of the subject of about 0.5 or less.

Prior to administration of the recombinant polypeptide, the subject may be characterized as having an average Pediatric Outcomes Data Collection Instrument (PODCI) score of about 40 or less. Administration of the recombinant polypeptide may result in an average PODCI score of the subject of about 40 or more.

Prior to administration of the recombinant polypeptide, the subject may be characterized as having an average Muscle Strength Grade of less than about 5. Administration of the recombinant polypeptide may result in an average increase in a Muscle Strength Grade of the subject of about 1 or more.

Prior to administration of the recombinant polypeptide, the subject may be characterized as having an average Hand Held Dynamometry (HHD) value of less than about 80% of a predicted HHD value. Administration of the recombinant polypeptide may result in an average HHD value of the subject of about 80% or more of a predicted HHD value. The HHD value may represent the grip strength, knee flexion, knee extension, hip flexion, hip extension, or hip abduction of the subject.

The subject may have a muscle weakness disease selected from muscular dystrophy, myasthenia gravis, and calcium pyrophosphate deposition disease (CPPD), amyotrophic lateral sclerosis (ALS), myositis, myotonic dystrophy, myotonia, Guillain-Barre syndrome, Duchenne muscular dystrophy (DMD), and Lambert-Eaton myasthenic syndrome.

In some embodiments of any of the methods of treatment and/or diagnosis described herein explicitly exclude subjects with familial hypophosphatemia (e.g., ADHR, autosomal recessive hypophosphatemic rickets, X-linked hypophosphatemic rickets, and XLH) or calcium pyrophosphate deposition disease (CPPD).

### Definitions

As used herein, “a” or “an” means “at least one” or “one or more” unless otherwise indicated. In addition, the singular forms “a,” “an,” and “the” include plural referents unless the context clearly dictates otherwise.

As used herein, “about” refers to an amount that is +10% of the recited value and is preferably #5% of the recited value, or more preferably +2% of the recited value.

By “asfotase alfa” is meant a human TNALP (hTNALP) fusion protein including a soluble glycoprotein of two identical polypeptide chains, in which each polypeptide chain includes amino acid residues 1-726 of SEQ ID NO: 1. The structure of each polypeptide chain includes the catalytic domain of hTNALP, the human immunoglobulin G1 Fc domain, and a deca-aspartate peptide used as a bone targeting domain (the structure hTNALP-Fc-D₁₀). The two polypeptide chains are covalently linked by two disulfide bonds. Asfotase alfa has been approved under the trade name STRENSIQ® (Alexion Pharmaceuticals, Inc., Boston, MA) in the United States, Europe, Japan, Canada, Israel, Australia, and Korea for the treatment of subjects with HPP.

The terms “individual,” “subject” and “patient” are used interchangeably and refer to any subject for whom diagnosis, treatment, or therapy is desired, particularly humans. As used herein, an “at risk” subject or a subject “being prone to” a disease is a subject who is identified as having a risk of developing a disease, disorder or symptoms associated with a muscle weakness disease.

As used herein, “average” refers to a numerical value expressing the mean or median of a data set. The mean of a data set is calculated by dividing the sum of the values in the data set by their number. The median of a data set is calculated by determining the middle value in a list data of odd numbers or by determining the mean of the two data values in the middle in a list of even numbers.

The term “wild-type” “or “wild-type sequence” used for TNALP or other genes or proteins in the instant disclosure refers to the typical form of such genes or proteins as found in nature in normal human, non-human mammals, or other living organisms. A wild-type sequence may refer to the standard “normal” allele at a locus for a gene or the standard “normal” primary amino acid sequence (optionally with the standard “normal” post-translational modifications to and/or inter-chain bonds and/or interactions among amino acid residues) for a polypeptide or protein, in contrast to that produced by a non-standard, “mutant” allele or amino acid sequence/modification/interaction. “Mutant” alleles can vary to a great extent, and even become the wild type if a genetic shift occurs within a population. It is now appreciated that most or all gene loci (and less frequently, but still possible, for most polypeptide sequences) exist in a variety of allelic forms, which vary in frequency throughout the geographic range of a species, and that a uniform wild type may not necessarily exist. In general, however, the most prevalent allele or amino acid sequence—i.e., the one with the highest frequency among normal individual human or other organisms—is the one deemed as wild type in the instant disclosure.

The term “normal subject” as used herein, refers to, except as specified otherwise, a subject, e.g., a human, without any evidence of a muscle weakness disease and/or symptoms or physiological consequences thereof caused by or related to, for example, aberrant alkaline phosphatase activity (which may be due to, e.g., deficient or lack of gene expression or protein levels and/or loss-of-function of gene or protein mutations). An example of a normal subject is a human lacking muscle weakness or muscle weakness symptoms and who exhibits no mutations or modifications to alkaline phosphatase genes or proteins (e.g., TNSALP) that may result in HPP-related muscle weakness. A normal subject may also be one exhibiting no aberrant endogenous alkaline phosphatase activity (which may be tested by, e.g., assessing the levels of PPi, PEA and PLP relative to a healthy control).

As used herein, an “elevated” or “increased” concentration refers to a concentration of a molecule (e.g., a substrate of TNSALP, such as PPi) in a subject having or being prone to a muscle weakness disease described herein that is higher than the concentration of the molecule in a normal subject, such as a subject without the muscle weakness disease, or in the same subject at a time point when the subject has no apparent symptoms of a muscle weakness disease (e.g., after treatment). An “elevated concentration” of the molecule can be determined in a cell, tissue, or organ of the subject, such as in the blood or serum of the subject.

The terms “Bayley Scales of Infant and Toddler Development, 3^{rd} Edition” or “BSID-III” as used herein refer to a standardized series of measurements used to assess the motor (fine and gross), language (receptive and expressive), and cognitive development of subjects. See Bayley, (2006). Bayley scales of infant and toddler development: administration manual. San Antonio, TX: Harcourt Assessment, hereby incorporated by reference in its entirety. The BSID-III measurements include a series of developmental play tasks to be administered to the subject. Raw scores of successfully completed items are converted to scaled scores. The scaled scores are then used to determine the subject's performance compared to healthy, age-adjusted subjects. The BSID-III can also include the Social-Emotional Adaptive Behavior Questionnaire, which is completed by the parent/guardian, to establish the range of adaptive behaviors of the subject. For example, measurements for determining the BSID-III score (e.g., the BSID-III gross motor function score) can include prehension, perceptual-motor integration, motor planning and speed, visual tracking, reaching, object grasping, object manipulation, functional hand skills, responses to tactile information, movement of the limbs and torso, static positioning, dynamic movement, balance, and motor planning. These subject measurements are then converted into a BSID-III scaled score (e.g., the BSID-III gross motor function scaled score) ranging from 0 to 14, in which scores of about 7 to about 13 are considered the normal range of healthy subjects.

The term “bone-targeting moiety,” as used herein, refers to an amino acid sequence of between 1 and 50 amino acid residues in length having a sufficient affinity to the bone matrix, such that the bone-targeting moiety, singularly, has an in vivo binding affinity to the bone matrix of about 10⁻⁶ M to about 10⁻¹⁵ M (e.g., 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, or 10⁻¹⁵ M).

The terms “Bruininks-Oseretsky Test of Motor Proficiency 2nd Edition” or “BOT-2,” as used herein, refer to the second edition of a standardized test of gross and fine motor performance for subjects, e.g., from about 4 to about 21 years of age. See Bruininks, R. H. (2005). Bruininks-Oseretsky Test of Motor Proficiency, (BOT-2). Minneapolis, MN: Pearson Assessment, hereby incorporated by reference in its entirety. The BOT-2 is administered individually to assess gross and fine motor skills of a range of subjects. In particular, the BOT-2 can be used to evaluate physical impairments and mobility restrictions in subjects having HPP. The BOT-2 provides composite BOT-2 scores in the following areas: strength, running speed and agility, fine motor precision, fine motor integration, manual dexterity, bilateral coordination, balance, and upper-limb coordination. For example, a BOT-2 strength score can be determined by having a subject perform sit-ups, v-ups, standing long jump, wall sit, and push-ups. A running speed and agility score can be determined by having a subject step over a balance beam or perform a shuttle run, two-legged side hop, or one-legged side hop. Both BOT-2 strength and BOT-2 running speed and agility scores range from 0 to 25, in which a score of about 10 to 20 is considered representative of healthy subjects.

The terms “Childhood Health Assessment Questionnaire” or “CHAQ,” as used herein refer to a questionnaire that is used to assess the health status (e.g., ability to perform activities of daily living (ADLs) and incidence of pain) of subjects of 1 to 19 years of age, such as subjects with an HPP-like disease. For a description of the CHAQ index, see Bruce & Fries (J. Rheumatol. 30(1): 167-178, 2003), hereby incorporated by reference in its entirety. The CHAQ may be administered by interview or self-report for children greater than 8 years of age. The CHAQ includes eight sub-scales for dressing/grooming, arising, eating, walking, hygiene, reach, grip, and activities. The range of scores within each category is from 0 to 3, in which a score of 0 indicates without any difficulty; a score of 1 indicates with some difficulty; a score of 2 indicates with much difficulty; and a score of 3 indicates that the subject is unable to perform the activity. The CHAQ index may also be used to determine the presence and severity of pain.

By “extracellular domain” is meant an extracellular portion of a native protein, e.g., alkaline phosphatase. In particular, the extracellular domain lacks a signal peptide.

By “Fc” is meant a fragment crystallizable region of an immunoglobulin, e.g., IgG-1, IgG-2, IgG-3, IgG-3, or IgG-4, including the CH2 and CH3 domains of the immunoglobulin heavy chain. Fc may also include any portion of the hinge region joining the Fab and Fc regions. The Fc can be from any mammal, including human, and may be post-translationally modified (e.g., by glycosylation). In a non-limiting example, Fc can be the fragment crystallizable region of human IgG-1 having the amino acid sequence of SEQ ID NO: 20.

By “fragment” is meant a portion of a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more of the entire length of the reference nucleic acid molecule or polypeptide. A fragment may contain, e.g., 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 400, 500, 600, 700, or more amino acid residues, up to the entire length of the polypeptide. Exemplary soluble alkaline phosphatase (sALP) fragments have amino acid residues 18-498, 18-499, 18-500, 18-501, 18-502, 18-503, 18-504, 18-505, 18-506, 18-507, 18-508, 18-509, 18-510, 18-511, or 18-512 of an ALP (e.g., SEQ ID NOs: 2-6), and may include additional C-terminal and/or N-terminal portions.

The terms “Hand Held Dynamometry” and “HHD” as used interchangeably herein refer to a method for measuring the grip and muscle strength of a subject, in particular, a subject having or being prone to a muscle weakness disease. A dynamometer can be used to assess grip strength, knee flexion, knee extension, hip flexion, hip extension, and hip abduction of a subject (e.g., a subject having or being prone to a muscle weakness disease). For example, knee flexion and extension and also hip flexion, extension, and abduction of a subject having or being prone to a muscle weakness disease can be measured using, e.g., a MICROFET2™ Dynamometer, while grip strength of the subject can be measured using, e.g., a Jamar Grip Dynamometer. In particular, the administrator holds the dynamometer stationary, and the subject exerts a maximal force against the dynamometer. Peak force data is collected in pounds, then converted to Newtons (N). Torque values are then calculated using limb length in N-meters. The torque value can then be compared to the value of, e.g., a normal subject of about the same age, the same gender, and/or the same height, and expressed as a percentage value to generate the HHD value of the subject.

The terms “hypophosphatasia” or “HPP,” as used herein, refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNALP). HPP may be further characterized as infantile HPP, childhood HPP, perinatal HPP (e.g., benign perinatal HPP or lethal perinatal HPP), or odonto-HPP.

The terms “hypophosphatasia-like disease” or “HPP-like disease,” as used herein, refer to diseases that present with similar symptoms as HPP, but are not caused by a loss-of-function mutation in the ALPL gene.

By “naïve patient” or “naïve subject” is meant a patient or subject having a muscle weakness disease described herein that has never received treatment with an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa).

By “pain” as used herein refers to physical suffering or discomfort caused by a muscle weakness disease described herein, such as muscle pain. For instance, symptoms of pain can include, e.g., soreness, tightness, or stiffness. The severity of pain can vary between subjects (e.g., chronic pain or acute pain). In particular, chronic pain refers to pain that lasts longer than three to six months or pain that extends beyond the expected period of healing. In contrast, acute pain refers to pain that typically lasts less than three to six months. As described herein, therapeutic compositions (e.g., including a sALP, such as asfotase alfa) can be administered to a subject suffering from pain (e.g., muscle pain) in an amount sufficient to relieve or at least partially relieve the symptoms of pain (e.g., discomfort, soreness, tightness, or stiffness) and its complications (e.g., fatigue, sleeplessness, weakened immune system, depression, anxiety, stress, irritability, or disability).

The terms “peptide,” “polypeptide,” and “protein” are used interchangeably and refer to any chain of two or more natural or unnatural amino acid residues, regardless of post-translational modification (e.g., glycosylation or phosphorylation), constituting all or part of a naturally-occurring or non-naturally occurring polypeptide or peptide, as is described herein.

By “pharmaceutically acceptable carrier” or “pharmaceutically acceptable excipient” is meant at least one carrier or excipient, respectively, that is physiologically acceptable to a subject (e.g., a human) and that retains the therapeutic properties of a molecule with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable carriers and their formulations are known to those skilled in the art and described, e.g., in Remington's Pharmaceutical Sciences (Remington: The Science and Practice of Pharmacy, 22nd Ed., Allen, Ed. 2012).

By “pharmaceutical composition” is meant a composition containing a polypeptide or nucleic acid molecule as described herein formulated with at least one pharmaceutically acceptable excipient, diluent, or carrier. The pharmaceutical composition may be manufactured or sold with the approval of a governmental regulatory agency as part of a therapeutic regimen for the treatment or prevention of a disease or event in a subject. Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form. In one embodiment, the pharmaceutical composition of the present disclosure is subcutaneously administered or is formulated for subcutaneous administration.

The term “physical impairments,” as used herein, refers to a physiological condition, such as bone weakness and muscle weakness diseases described herein that can restrict or eliminate, e.g., ambulation, functional endurance, and ability to perform activities of daily living (ADL) of a subject. In particular, physical impairments may restrict or eliminate a subject's ability to perform ADL, which are routine activities that healthy subjects perform on a daily basis without requiring assistance, such as functional mobility or transferring (e.g., walking), bathing and showering, dressing, self-feeding, and personal hygiene and grooming. As described herein, therapeutic compositions (e.g., compositions including a sALP, such as asfotase alfa) can be administered to a subject to decrease the severity and/or frequency of physical impairments associated with muscle weakness.

The terms “Pediatric Outcomes Data Collection Instrument” or “PODCI,” as used herein, refer to a questionnaire used to assess overall health, incidence of pain, and ability to perform ADLs of subjects under 19 years of age, particularly in subjects with chronic health disorders, such as a subject with an HPP-like disease. For a description of the PODCI, see Plint et al. (J. Pediatr. Orthop. 23(6): 788-790, 2003), hereby incorporated by reference in its entirety. The questionnaire may be completed by the subject or by a parent/guardian of the subject with knowledge of the subject's condition. The eight scales generated from the PODCI include the following: 1) the upper extremity and physical function scale to measure difficulty encountered in performing daily personal care and student activities; 2) the transfer and basic mobility scale to measure difficulty experienced in performing routine motion and motor activities in daily activities; 3) the sports/physical functioning scale to measure difficulty or limitations encountered in participating in more active activities or sports; 4) the pain/comfort scale to measure the level of pain experienced during the past week; 5) the treatment expectations scale to measure the long term expectations of treatment; 6) the happiness scale to measure overall satisfaction with personal looks and sense of similarity to friends and others of own age; 7) the satisfaction with symptoms scale to measure the subject's acceptance of current limitations should this be a life-long state; and 8) the global functioning scale, which is a general combined scale calculated from the first four scales listed above. Standardized scores are generated from a series of questions in the PODCI and converted to a 0 to 100 scale, in which 0 represents significant disability and 100 represents less disability.

The terms “Peabody Developmental Motor Scales, 2nd Edition” or “PDMS-2,” as used herein, refer to an early childhood motor development program that provides an assessment of gross and fine motor skills in subjects from birth throughout childhood (e.g., infants and children). For a description of the PDMS-2 scales, see van Hartingsveldt et al. (Occup. Ther. Int. 12(1): 1-13, 2005), hereby incorporated by reference in its entirety. The PDMS-2 is composed of six subtests that measure interrelated motor abilities of early development. The six subtests include the following: 1) the locomotor subtest to measures a subject's ability to move from one place to another (measurements include crawling, walking, running, hopping, and jumping forward); 2) the reflexes subtest to measure a subject's ability to automatically react to environmental events; 3) the stationary subtest to measure a subject's ability to sustain control of his or her body within the center of gravity and retain equilibrium; 4) the object manipulation subtest to measure a subject's ability to manipulate an object, such as catching, throwing, and kicking a ball; 5) the grasping subtest to measure a subject's ability to use his or her hands, such as the ability to hold an object with one hand and actions involving the controlled use of the fingers of both hands; and 6) the visual-motor integration subtest to measure a subject's ability to use his or her visual perceptual skills to perform complex eye-hand coordination tasks, such as reaching and grasping for an object, building with blocks, and copying designs. The PDMS-2 measurements for each subtest is converted into a PDMS-2 score, such as the PDMS-2 locomotor standard score ranging from 0 to 13, in which the range of a healthy subject is from about 7 to about 13.

The terms “sALP,” “soluble alkaline phosphatase,” and “extracellular domain of an alkaline phosphatase” are used interchangeably and refer to a soluble, non-membrane-bound alkaline phosphatase or a domain, biologically active fragment, or biologically active variant thereof. sALPs include, for example, an alkaline phosphatase lacking a C-terminal glycolipid anchor (GPI signal sequence, e.g., polypeptides including or consisting of the amino acid residues 18-502 of a human TNALP (SEQ ID NOs: 2, 3, 4, 5, or 6)). In particular, a TNALP may include, e.g., a polypeptide including or consisting of amino acid residues 1-485 of SEQ ID NO: 1, such as asfotase alfa, or a polypeptide variant having at least 95% sequence identity to the amino acid residues 1-485 of SEQ ID NO: 1 (SEQ ID NO: 21 has 95.8% sequence identity with SQ ID NO: 1 and SEQ ID NO: 22 has 96.08% sequence identity with SEQ ID NO. 1). sALPs further include, for example, mammalian orthologs of human TNALP, such as a rhesus TNALP (SEQ ID NO: 7), a rat TNALP (SEQ ID NO: 8), a canine TNALP (SEQ ID NO: 9), a porcine TNALP (SEQ ID NO: 10), a murine TNALP (SEQ ID NO: 11), a bovine TNALP (SEQ ID NOs: 12-14), or a feline TNALP (SEQ ID NO: 15). SALPs also include soluble, non-membrane-bound forms of human PALP (e.g., polypeptides including or consisting of amino acid residues 18-502 of SEQ ID NOs: 16 or 17), GCALP (e.g., polypeptides including or consisting of amino acid residues 18-502 of SEQ ID NO: 18), and IALP (e.g., polypeptides including or consisting of amino acid residues 18-502 of SEQ ID NO: 19), and additional variants and analogs thereof that retain alkaline phosphatase activity, e.g., the ability to hydrolyze PPi. A sALP, in particular, lacks the N-terminal signal peptide (e.g., aa 1-17 of SEQ ID NOs: 2-6, 8, 11-13, or 15 or aa 1-25 of SEQ ID NO: 7).

By “sALP polypeptide” is meant a polypeptide having the structure A-sALP-B, wherein sALP is as defined herein and each of A and B is absent or is an amino acid sequence of at least one amino acid. An exemplary sALP polypeptide has an amino acid sequence comprising or consisting of the amino acids 1-485 of SEQ ID NO: 1. Other exemplary sALP polypeptides include any sALP fusion polypeptides described herein (for example the sALP fusion polypeptide of SEQ ID NO: 1 or a polypeptide variant having at least 95% sequence identity to the sequence of SEQ ID NO: 1, e.g., asfotase alfa).

By “signal peptide” is meant a short peptide (5-30 amino acids long) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the Golgi apparatus. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal peptide can be one that is, for example, substantially identical to amino acid residues 1-17 of SEQ ID NOs: 2-6 or amino acid residues 1-25 of SEQ ID NO: 7.

As used herein, when a polypeptide or nucleic acid sequence is referred to as having “at least X % sequence identity” to a reference sequence, wherein “X” is a real number, it is meant that at least X percent of the amino acid residues or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al., J. Mol. Biol. 215:403-10, 1990). These and other alignment algorithms are accessible using publicly available computer software such as “Best Fit” (Smith and Waterman, Advances in Applied Mathematics, 482-489, 1981) as incorporated into GeneMatcher Plus (Schwarz and Dayhoff, Atlas of Protein Sequence and Structure, Dayhoff, M. O., Ed pp 353-358, 1979), BLAST, BLAST-2, BLAST-P, BLAST-N, BLAST-X, WU-BLAST-2, ALIGN, ALIGN-2, CLUSTAL, Megalign (DNASTAR), or other software/hardware for alignment. In addition, those skilled in the art can determine appropriate parameters for measuring alignment, including any algorithms needed to achieve optimal alignment over the length of the sequences being compared.

By “therapeutically effective amount” is meant an amount of a polypeptide or nucleic acid molecule described herein that is sufficient to substantially improve, treat, prevent, delay, suppress, or arrest at least one symptom of muscle weakness. A therapeutically effective amount of a composition described herein may depend on the severity of the disorder being treated and the condition, weight, and general state of the subject and can be determined by an ordinarily-skilled artisan with consideration of such factors. A therapeutically effective amount of a composition described herein can be administered to a subject in a single dose or in multiple doses administered over a period of time.

By “treating,” “treat,” or “treatment” is meant the medical management of a subject with the intent to cure, ameliorate, stabilize, reduce the likelihood of, or prevent a muscle weakness diseases (e.g., in a subject with hypotonia) and/or management of a subject exhibiting or likely to have a muscle weakness diseases (e.g., in a subject with hypotonia), e.g., by administering a pharmaceutical composition (e.g., a sALP, such as asfotase alfa).

This term includes active treatment, that is, treatment directed specifically toward the improvement or associated with the cure of a disease, pathological condition, disorder, or event, and also includes causal treatment, that is, treatment directed toward removal of the cause of the associated disease, pathological condition, disorder, or event. In addition, this term includes palliative treatment, that is, treatment designed for the relief or improvement of at least one symptom rather than the curing of the disease, pathological condition, disorder, or event; symptomatic treatment, that is, treatment directed toward constitutional symptoms of the associated disease, pathological condition, disorder, or event; preventative treatment, that is, treatment directed to minimizing or partially or completely inhibiting the development of the associated disease, pathological condition, disorder, or event, e.g., in a subject who is not yet ill, but who is susceptible to, or otherwise at risk of, a particular disease, pathological condition, disorder, or event; and supportive treatment, that is, treatment employed to supplement another specific therapy directed toward the improvement of the associated disease, pathological condition, disorder, or event.

As used herein, “walking ability” refers to the ability of a subject (e.g., a subject having a muscle weakness disease described herein) to lift and set down each foot in turn. Walking ability may be assessed by tests, in particular, the Six-Minute Walk Test (6MWT). See the American Thoracic Society statement: guidelines for the six-minute walk test (American Journal of Respiratory and Critical Care Medicine, 166(1):111-7, 2002), hereby incorporated by reference in its entirety.

### BRIEF DESCRIPTION OF THE DRAWINGS

FIG. 1 is a graph showing RNA sequencing results of ATP1A3 transcripts. The x-axis represents position in the transcript and the y-axis is number of reads. Results are shown for patient A, mother, father, and three siblings.

FIG. 2A is a graph showing ALPL transcription of patient A (proband), mother, father, and three siblings. The transcription is measured in transcripts per million (TPM).

FIG. 2B is a graph of a Western Blot showing TNAP translation of patient A (proband), mother, father, and three siblings. Vinculin is used as a standard.

FIG. 3 illustrates the seahorse mitochondrial respiratory stress test of age and sex-matched HPP and WT mice extensor digitorum longus (EDL) muscle fibers

FIG. 4 illustrates spare respiratory capacity in HPP mice muscle after in vivo treatment with either vehicle or SEQ ID NO: 21

FIG. 5 illustrates a path for branched chain amino acids (BCAAs) in adenosine triphosphate (ATP) production via the electron transport chain (ETC) and oxidative phosphorylation (Ox-Phos);

FIG. 6, FIG. 7, and FIG. 8 illustrate BCAAs valine, leucine, and isoleucine levels, respectively, in the tibialis muscle of tissue nonspecific alkaline phosphatase (TNSALP) knock-out mice (Akp2−/− (KO) mice) treated with SEQ ID NO: 1 for 35 days followed by vehicle for 12 days (SEQ ID NO: 1 withdrawal), or treated continuously with SEQ ID NO: 1 for 47 days, as well as wild-type, untreated mice.

### DETAILED DESCRIPTION

Muscle weakness is a prominent symptom of several diseases and disorders. One such disease is hypophosphatasia (HPP), a congenital defect caused by loss-of-function mutations in the ALPL gene, which encodes tissue nonspecific alkaline phosphatase (TNSALP). Asfotase alfa (STRENSIQ®, Alexion Pharmaceuticals, Inc.), a TNSALP fusion protein, is the first and only treatment available to subjects diagnosed with HPP. Surprisingly, we discovered that subjects that do not have a loss-of-function mutation in the ALPL gene, yet exhibit similar symptoms as HPP, including muscle weakness (e.g., hypotonia), also respond to asfotase alfa treatment. Accordingly, featured are methods of treating muscle weakness in a subject without a loss-of-function mutation in ALPL that is characterized as having reduced concentration of TNSALP (e.g., in their serum) by administering a recombinant polypeptide having alkaline phosphatase activity (e.g., asfotase alfa).

Subjects that can be Treated Using the Compositions and Methods Described Herein

Featured are methods of treatment for muscle weakness in a subject with reduced levels of TNSALP by administering a polypeptide having alkaline phosphatase activity. The subject is one that does not have a loss-of-function mutation in ALPL, but may have a mutation in non-coding regions of the ALPL gene. The reduced level of TNSALP may be caused by one or more factors, such as reduced (e.g., reduced by 10%, 20%, 30%, 40%, 50%, 60%, 70%, 80%, 90%, or 100%) transcription of ALPL, reduced (e.g., reduced by 10%, 20%, 30%, 40%, 50%, 60%, 70%, 80%, 90%, or 100%) translation of ALPL mRNA, elevated (e.g., elevated by 10%, 20%, 30%, 40%, 50%, 60%, 70%, 80%, 90%, or 100%) or reduced (e.g., reduced by 10%, 20%, 30%, 40%, 50%, 60%, 70%, 80%, 90%, or 100%) posttranslational modification of TNSALP, and/or reduced (e.g., reduced by 10%, 20%, 30%, 40%, 50%, 60%, 70%, 80%, 90%, or 100%) TNSALP enzymatic activity. Reduced transcription of ALPL, reduced translation of ALPL mRNA, elevated or reduced posttranslational modification of TNSALP, and/or reduced TNSALP enzymatic activity may be measured with respect to an average, a normalized value, a baseline, a subject value before disease onset, or a healthy subject.

Loss-of-function mutations in ALPL are known in the art and are disclosed, e.g., in PCT Publication WO2011134084, the disclosure of which is hereby incorporated by reference in its entirety. Accordingly, featured are methods for treating subjects with muscle weakness that do not have one of the mutations described therein or otherwise known. A mutation that is not loss-of-function, e.g., a mutation in regulatory regions, 3′ or 5′ UTRs, enhancers, promoters, or intronic regions of ALPL, may contribute to a reduced level of TNSALP. Alternatively, or in addition, the subject may have a mutation (e.g., single nucleotide polymorphism(s) (SNPs)) anywhere in their genomes, such as in regulatory regions, 3′ or 5′ UTRs, enhancers, promoters, or intronic regions of non-ALPL genes.

The subject may have one or more structural variants in their genome that produce reduced TNSALP activity leading to muscle weakness. Examples of structural variants include copy number variants, insertions, duplications, inversions, translocations, and breakends. The subject may have a mutation or structural variant in a coding region(s) for a protein, enzyme, or regulatory RNA (e.g., noncoding RNA) other than TNSALP. For example, other proteins or RNAs that interact with TNSALP or the ALPL gene that directly or indirectly modulate the function, activity, and/or expression of TNSALP may have a mutation or structural variant that imparts a downstream effect that reduces TNSALP level. For example, the subject may have a mutation or structural variant in one or more of the ATP1A3, ANKH, ENPP1, FGFR3, PHOSPHO1, PTHIR, PTH2R, SPP1, TNFRSF11A, TNFRSF11B, COL1A1, COL1A2, SOX9, PDXP, AOX1, PNPO, PDXK, ADCK3, MTRNR2, or S1PR1 genes. The mutation or structural variant may be in a coding region, 5′ UTR, 3′UTR, or intronic sequence. The subject may have an ATP1A3 variant c.357+1G>A. The subject may have an ADCK3 variant m. 1665G>A. The subject may have an MTRNR2 variant m.1836A>G.

The ATP1A3 gene encodes an alpha-subunit of the Na+/K+ ATPase pump that uses energy from ATP to transport ions into and out of cells to maintain electrochemical gradient. It also plays a role in neuronal function, muscle contraction, and the reuptake of neurotransmitters in the central nervous system. All reported deleterious ATP1A3 mutations display a dominant mode of inheritance with variable penetrance. Furthermore, deleterious mutations are associated with rapid-onset dystonia-Parkinsonism phenotype characterized by motor delay and ataxia and alternating hemiplegia of childhood, and cognitive problems. The phenotype can vary in children and may include hypotonia, ataxia, difficulty speaking, and slurred speech. Subjects with mutations in ATP1A3 may exhibit decreased levels of ALP or symptoms associated therewith as described herein. A subject with a mutation in ATP1A3 having muscle weakness may benefit from treatment with a polypeptide having alkaline phosphatase activity according to the methods described herein.

The ADCK3 gene encodes a mitochondrial protein, which functions in an electron-transferring membrane protein complex in the respiratory chain. Mutations in this gene impair Coenzyme Q10 production. PLP is also a required cofactor for enzymes in the biosynthesis pathway of Q10. Mutations in the ADCK3 gene are associated with autosomal-recessive cerebellar ataxias. Subjects with mutations in ADCK3 may exhibit decreased levels of ALP or symptoms associated therewith as described herein. A subject with a mutation in ATP1A3 having muscle weakness may benefit from treatment with a polypeptide having alkaline phosphatase activity according to the methods described herein.

A subject treated according to the methods described herein may be characterized as having one or more of the following: an elevated PPi concentration, decreased alkaline phosphatase concentration and/or activity, an average BOT-2 strength score of, e.g., less than 10, an average BOT-2 running speed and agility score of, e.g., less than 5, an average CHAQ index score of, e.g., greater than about 0.8, or an average PODCI score of, e.g., less than about 40, an average 6MWT of, e.g., less than about 80% of the predicted 6MWT value (e.g., in which the predicted 6MWT value is the 6MWT value of an age-matched and/or gender-matched normal subject), a Muscle Strength Grade of, e.g., less than 5, and/or an average HHD value (e.g., an average HHD muscle or grip strength value) of, e.g., less than about 50% of the predicted HHD value (e.g., in which the predicted HHD value is the HHD value of an age-matched and/or gender-matched normal subject). In particular, the subject may be one that has been identified as having or being prone to a muscle weakness.

The subject may be identified as being in need of treatment. For example, subjects identified as having or being prone to a muscle weakness disease (e.g., a disease not caused by a loss-of-function mutation in ALPL but characterized by, e.g., reduced ALP levels or activity or increased ALP substrate levels). Such a subject may be treated by administration of a recombinant polypeptide having alkaline phosphatase activity (such as TNALP, for example the sALP polypeptide of SEQ ID NO: 1 or a polypeptide variant having at least 95% sequence identity to the sequence of SEQ ID NO: 1, e.g., asfotase alfa). The subject may be characterized as having an elevated PPi concentration, decreased ALP concentration, an average BOT-2 strength score of, e.g., less than 10, an average BOT-2 running speed and agility score of, e.g., less than 5, an average CHAQ index score of, e.g., greater than about 0.8, an average PODCI score of, e.g., less than about 40, an average 6MWT of, e.g., less than about 80% of the predicted 6MWT value, a Muscle Strength Grade of, e.g., less than 5, and/or an average HHD value (e.g., an average HHD muscle or grip strength value) of, e.g., less than about 80% of the predicted HHD value. For example, an elevated concentration of PPi in a sample (e.g., a plasma sample) from an infant or child (e.g., a subject less than about 12 years of age) of about 5.71 μM or greater, an elevated concentration of PPi in a sample (e.g., a plasma sample) from an adolescent (e.g., a subject of about 13 to about 18 years of age) of about 4.78 μM or greater; and an elevated concentration of PPi in a sample (e.g., a plasma sample) from an adult (e.g., a subject of greater than about 18 years of age) of about 5.82 μM or greater can be used to identify these subjects as being in need of treatment. In particular, a decreased ALP concentration in a sample (e.g., a plasma sample) from the subject of, e.g., about 90 U/L or less for a subject of 0 to 14 days of age; about 134 U/L or less for a subject of 15 days of age to less than 1 year of age; about 156 U/L or less for a subject of about 1 year of age to less than 10 years of age; about 141 U/L or less for a subject of about 10 years of age to less than about 13 years of age; about 62 U/L or less for a female subject of about 13 years of age to less than about 15 years of age; about 127 U/L or less for a male subject of about 13 years of age to less than about 15 years of age; about 54 U/L or less for a female subject of about 15 years of age to less than about 17 years of age; about 89 U/L or less for a male subject of about 15 years of age to less than about 17 years of age; about 48 U/L or less for a female subject of about 17 years of age or older; or about 59 U/L or less for a male subject of about 17 years of age or older can be used to identify these subjects as being in need of treatment.

The subject having or being prone to a muscle weakness disease (e.g., subjects without a loss-of-function mutation in ALPL) may be identified by an elevated PPi concentration, a decreased alkaline phosphatase concentration, and/or a decreased grip or muscle strength (e.g., as assessed using the BOT-2, 6MWT, CHAQ, PODCI, Muscle Strength Grade, and/or HHD). The subject may have reduced transcription of ALPL, reduced translation of ALPL mRNA, elevated or reduced posttranslational modification of TNSALP, and/or reduced TNSALP enzymatic activity, e.g., relative to an average, a normalized value, a baseline, a subject value before disease onset, or a healthy subject.

Also featured are methods of identifying a subject having or being prone to a muscle weakness disease, in which the subject has elevated PPi concentrations, decreased ALP concentrations, and/or decreased grip or muscle strength; and then treating, ameliorating, or reducing the risk of at least one symptom of the muscle weakness disease in the subject, e.g., by administration of an ALP (e.g., asfotase alfa).

The subject can be identified irrespective of whether they have previously been diagnosed with hypophosphatasia (HPP), calcium pyrophosphate deposition disease (CPPD), or familial hypophosphatemia (such as autosomal dominant hypophosphatemic rickets (ADHR), autosomal recessive hypophosphatemic rickets, X-linked hypophosphatemic rickets, X-linked hypophosphatemia (XLH), etc.). The subject is identified, for example, based on an elevated PPi concentration. Causes of elevated PPi concentration include, for example, defects in signaling molecules, or mutations in genes that encode such signaling molecules, which regulate the production, degradation, or the stability of PPi. For example, the defects or mutations to signaling molecules may result in overexpression of PPi or decreased degradation or hydrolysis of PPi. The defects in signaling molecules also include defects in the co-factors or other molecules facilitating the function of the signaling molecules. For example, in CPPD, a deficiency of Mg, which acts as a cofactor for various phosphatases, leads to elevated levels of PPi.

### Targeted Muscle Weakness Diseases

Muscle weakness, myopathies, and myasthenia that can be treated by the methods described herein include any disease or disorder which causes, is due to, or is related to at least one symptom of muscle weakness. The terms “muscle weakness, “myopathy,” and “myasthenia,” or other similar expressions in this disclosure, refer to a condition related to impaired status of muscle function, such as a lack or defect of muscle strength, as compared to other subjects lacking the condition or relative to the same subject at the time point prior to having the condition. Muscle weakness can be divided into conditions that have either true or perceived muscle weakness. True muscle weakness may include a condition where the force exerted by the muscles is less than would be expected. For example, true muscle weakness includes a variety of skeletal muscle diseases, including muscular dystrophy and inflammatory myopathy. Other examples include neuromuscular junction disorders, such as myasthenia gravis. Muscle weakness can also be caused by low levels of potassium and other electrolytes within muscle cells, where the force exerted by the muscles is less than would be expected. Perceived muscle weakness (or non-neuromuscular weakness) describes a condition where a subject feels more effort than normal (i.e., compared to other subjects lacking the condition or to the same subject at the time point prior to having the condition) is required to exert a certain amount of force but actual muscle strength is normal, for example chronic fatigue syndrome.

In some conditions, such as myasthenia gravis, muscle strength is normal when resting, but true weakness occurs after the muscle has been subjected to exercise. This is also true for some cases of chronic fatigue syndrome, where objective post-exertion muscle weakness with delayed recovery time has been measured and is a feature of some of the published definitions. These diseases or disorders are also examples of a “muscle weakness disease” within the scope of this disclosure.

Muscle weakness can also be classified as either “proximal” or “distal” based on the location of the muscles that it affects. Proximal muscle weakness affects muscles closest to the body's midline, while distal muscle weakness affects muscles further out on the limbs. Proximal muscle weakness can be seen in Cushing's Syndrome and hyperthyroidism.

Other categories of muscle weakness exist in practice. For example, neuromuscular fatigue can be classified as either “central” or “peripheral” depending on its cause. Central muscle fatigue manifests as an overall sense of energy deprivation, while peripheral muscle fatigue manifests as a local, muscle-specific inability to do work

The severity of muscle weakness can be classified into different “grades” based on the following exemplary criteria:
Grade 0: No contraction or muscle movement.
Grade 1: Trace of contraction, but no movement at the joint.
Grade 2: Movement at the joint with gravity eliminated.
Grade 3: Movement against gravity, but not against added resistance.
Grade 4: Movement against external resistance with less strength than usual.
Grade 5: Normal strength.
   A subject classified as having a muscle weakness disorder within any of Grades 0-4 may be treated according to the methods disclosed herein.

### Hypophosphatasia (HPP) and Muscle Weakness

Hypophosphatasia (HPP) is the rare inherited metabolic disorder resulting from loss-of-function mutation(s) in the tissue-nonspecific alkaline phosphatase (TNSALP) gene. We have identified a subset of subjects who were diagnosed with HPP yet, upon sequencing analysis, do not exhibit a loss-of-function mutation in ALPL, which encodes TNSALP. Thus, some other mechanism is contributing to the onset of HPP-like symptoms. A biochemical hallmark in these subjects is subnormal ALP activity in serum (hypophosphatasemia), which leads to elevated blood and/or urine levels of three phosphocompound substrates: inorganic pyrophosphate (PPi), phosphoethanolamine (PEA) and pyridoxal 5′-phosphate (PLP). TNSALP deficiency can cause a spectrum of sequelae including premature loss of primary teeth, rickets, poor growth, muscle weakness, compromised physical function, and pain. Asfotase alfa treatment dramatically improves bone mineralization in subjects with HPP, yet it was uncertain whether it would be efficacious in treating muscle weakness. As described herein, we have discovered that asfotase alfa has a therapeutic effect on the muscles of a subject (e.g., a subject having a muscle weakness disease, such as muscle weakness in a subject having an HPP-like disease), even in subjects without a loss-of-function mutation in ALPL.

### Calcium Pyrophosphate Deposition Disease (CPPD or CPDD) and Muscle Weakness

Calcium pyrophosphate deposition disease (CPPD or CPDD), or calcium pyrophosphate dihydrate crystal deposition disease, is a metabolic arthropathy caused by the deposition of calcium pyrophosphate dihydrate crystals in and around joints, especially in articular cartilage and fibrocartilage. These diseases may be present in a subject without a loss-of-function mutation in ALPL and who exhibit a reduced level of TNSALP. Although CPPD is often asymptomatic, with only radiographic changes seen (i.e., chondrocalcinosis), various clinical manifestations may occur, including acute (pseudogout) and chronic arthritis. The crystal deposits provoke inflammation in the joint, which can cause the joint cartilage to break down. The disease may take a few different arthritis-related forms: osteoarthritis, a chronic rheumatoid arthritis (RA)-like inflammatory arthritis, or an acutely painful inflammatory condition called pseudogout. The name pseudogout comes from the fact that it resembles another acutely painful condition called gout. The main difference is the type of crystals involved in the inflammation and damage. Almost any joint may be affected by CPPD, although the knees, wrists, and hips are most often affected. This condition is the most common cause of secondary metabolic osteoarthritis. A subject with CPPD can experience significant morbidity due to the pain of an acute attack of pseudogout or to symptoms of chronic arthropathy. Treatment of symptomatic CPPD is important to prevent further end-organ damage, but it cannot reverse the joint disease.

The exact mechanism for the development of CPPD remains unclear. From aging, genetic factors, or both, subjects have increased adenosine triphosphate breakdown resulting in increased inorganic pyrophosphate concentration in the joints. Changes in the cartilage matrix may play an important role in promoting calcium pyrophosphate dihydrate crystal deposition. Over activity of enzymes that break down triphosphates, such as nucleoside triphosphate pyrophosphohydrolase, has been observed in the cartilage of subjects with CPPD. Therefore, inorganic pyrophosphate can bind calcium, leading to deposition in the cartilage and synovium. Hyaline cartilage is affected most commonly, but fibrocartilage, such as the meniscal cartilage of the knee, can also be involved (Pritzker et al., 1988 J Rheumatol. 15(5):828-835).

### Other Diseases and Muscle Weakness

Similarly to HPP and CPPD (or CPDD), other diseases or disorders may include at least one symptom of muscle weakness. Among them, some types of muscle weakness diseases have characteristic elevated inorganic pyrophosphate (PPi) concentration. These muscle weakness diseases with elevated PPi concentration are also targets for treatment with asfotase alfa in the instant disclosure.

For example, subjects diagnosed with familial hypophosphatemia (such as autosomal dominant hypophosphatemic rickets (ADHR), autosomal recessive hypophosphatemic rickets, X-linked hypophosphatemic rickets, and X-linked hypophosphatemia (XLH), etc.) are typically characterized as having a muscle weakness phenotype. Hypophosphatemia, or hypophosphatemic rickets, is a form of rickets that is characterized by low serum phosphate levels and resistance to treatment with ultraviolet radiation or vitamin D ingestion. X-linked hypophosphatemia (XLH) is a dominant disorder and accounts for more than 80% of all familial hypophosphatemia. XLH is considered to be a systemic disorder, from mutation of the phosphate-regulating gene homologous to endopeptidases on the X chromosome (PHEX). XLH subjects demonstrate a normal or low serum concentration of 1,25-dihydroxyvitamin D3, suggestive of inadequate formation of this vitamin D metabolite. The remaining 20% of familial hypophosphatemia subjects have autosomal dominant hypophosphatemic rickets from gain-of-function autosomal recessive hypophosphatemic rickets and hereditary hypophosphatemic rickets with hypercalciuria.

Other diseases characterized by weakness disease that may be treated according to the methods described herein include, for example, myasthenia gravis, amyotrophic lateral sclerosis (ALS), myositis, myotonic dystrophy, myotonia, Guillain-Barre syndrome, Duchenne muscular dystrophy (DMD), and Lambert-Eaton myasthenic syndrome.

### Methods of Treatment

Provided herein are methods for treating or ameliorating at least one symptom of a subject (e.g., a child, adolescent, or adult, e.g., a subject who does not have a loss-of-function mutation in ALPL and has a reduced level of TNSALP) who has or is prone to a muscle weakness disease. Such treatment may include administering an alkaline phosphatase, or a polypeptide having alkaline phosphatase activity, to decrease the elevated PPi concentration in such subject. For example, a soluble alkaline phosphatase (sALP, such as TNALP, for example the sALP polypeptide of SEQ ID NO: 1 or a polypeptide variant having at least 95% sequence identity to the sequence of SEQ ID NO: 1, e.g., asfotase alfa) may be administered to children, adolescents, or adults.

Subjects may be diagnosed with a muscle weakness disease (such as HPP or HPP-like disease, CPPD, familial hypophosphatemia described herein, etc.) prior to administration of an alkaline phosphatase, or a polypeptide having alkaline phosphatase activity, (e.g., a sALP, such as TNALP, for example the sALP polypeptide of SEQ ID NO: 1 or a polypeptide variant having at least 95% sequence identity to the sequence of SEQ ID NO: 1, e.g., asfotase alfa). Additionally, a subject having or being prone to a muscle weakness disease can be a naïve subject that has not been previously treated with a sALP (such as TNALP, for example the sALP polypeptide of SEQ ID NO: 1 or a polypeptide variant having at least 95% sequence identity to the sequence of SEQ ID NO: 1, e.g., asfotase alfa). The subject may have a muscle weakness disease other than HPP, CPPD, or familial hypophosphatemia.

The method includes administering an alkaline phosphatase or a polypeptide having alkaline phosphatase activity (e.g., a sALP, such as TNALP, for example the sALP polypeptide of SEQ ID NO: 1 or a polypeptide variant having at least 95% sequence identity to the sequence of SEQ ID NO: 1, e.g., asfotase alfa) to a subject having or being prone to a muscle weakness disease in a single or multiple dosages over a period of time. In particular, a sALP, such as asfotase alfa, can be administered to a subject previously determined to have elevated inorganic pyrophosphate (PPi) concentration or at least one predetermined biomarker/score for muscle weakness, such as an average BOT-2 strength score of less than 10, an average BOT-2 running speed and agility score of less than 5, an average CHAQ index score greater than about 0.8, and/or an average PODCI score of less than about 40, an average 6MWT of less than about 80% of the predicted 6MWT value, a Muscle Strength Grade of less than 5, and/or an average HHD value (e.g., an average HHD muscle or grip strength value) of, e.g., less than about 80% of the predicted HHD value. For example, a sALP can be administered to a subject previously determined to have a concentration of PPi in a sample (e.g., a plasma sample) of greater than about 5.71 μM for an infant or child (e.g., a subject less than about 12 years of age); greater than about 4.78 μM for an adolescent (e.g., a subject of about 13 to about 18 years of age); or greater than about 5.82 μM for an adult (e.g., a subject of greater than about 18 years of age). In other embodiments, the muscle weakness disease described herein is caused by an elevated concentration of at least one alkaline phosphatase substrate (e.g., PPi, PLP, PEA, etc.). Alternatively, an alkaline phosphatase, or a polypeptide having alkaline phosphatase activity, (e.g., a sALP, such as TNALP, for example the sALP polypeptide of SEQ ID NO: 1 or a polypeptide variant having at least 95% sequence identity to the sequence of SEQ ID NO: 1, e.g., asfotase alfa) can be administered to a subject having or being prone to a muscle weakness disease prior to determination of muscle weakness score (e.g., using the BOT-2 strength score, BOT-2 running speed and agility score, the CHAQ index score, the BSID-III scaled score, the PDMS-2 standard score, a Muscle Strength score, a 6MWT value, and/or a HHD value). Treatment with an ALP according to the methods described herein promotes, e.g., an increase in activities of ADL, a decrease in pain, and/or an improvement in motor development.

Additionally, each of the described scores (e.g., the BOT-2 strength score, BOT-2 running speed and agility score, the CHAQ index score, the BSID-III scaled score, the PDMS-2 standard score, 6MWT, the 12-POMA-G, a modified performance-oriented mobility assessment (mPOMA-G, such as the one illustrated in Phillips et al. 2015 Bone Abstracts 4: P136), or the HHD value) of a subject having or being prone to a muscle weakness disease described herein can be used singly or in any combination to assess treatment efficacy using a sALP (such as TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the sALP is effective for treating the muscle weakness disease.

For example, when administration of an alkaline phosphatase or a polypeptide having alkaline phosphatase activity (e.g., a sALP, such as TNALP, for example the sALP polypeptide of SEQ ID NO: 1 or a polypeptide variant having at least 95% sequence identity to the sequence of SEQ ID NO: 1, e.g., asfotase alfa) to a subject having or being prone to a muscle weakness disease results in an average increase in the BOT-2 strength score to about 10 or greater than about 10, in which the subject previously had an average BOT-2 strength score of less than about 10, then the alkaline phosphatase or a polypeptide having alkaline phosphatase activity treatment is effective at treating, e.g., physical impairments associated with a muscle weakness disease. Alternatively, when administration of a sALP does not result in an average increase in the BOT-2 strength score to about 10 or greater than about 10, the dosage and/or frequency of alkaline phosphatase or a polypeptide having alkaline phosphatase activity administration can be changed in order to determine the effective amount of the alkaline phosphatase or a polypeptide having alkaline phosphatase activity for the subject. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

Additionally, when administration of an alkaline phosphatase or a polypeptide having alkaline phosphatase activity (e.g., a sALP, such as TNALP, for example the sALP polypeptide of SEQ ID NO: 1 or a polypeptide variant having at least 95% sequence identity to the sequence of SEQ ID NO: 1, e.g., asfotase alfa) to a subject having or being prone to a muscle weakness disease results in an improvement in the Muscle Strength Grade categorization of the subject of one or more (e.g., an improvement to a Muscle Strength Grade of 1, 2, 3, 4, or 5 from a prior, lower Muscle Strength Grade), in which the subject previously had an average Muscle Strength Grade of less than about 5, then the alkaline phosphatase or a polypeptide having alkaline phosphatase activity treatment is effective at treating, e.g., physical impairments associated with a muscle weakness disease. Alternatively, when administration of a sALP does not result in an improvement in the Muscle Strength Grade categorization of the subject of one or more from a prior, lower Muscle Strength Grade, the dosage and/or frequency of alkaline phosphatase or a polypeptide having alkaline phosphatase activity administration can be changed (e.g., increased) in order to determine the effective amount of the alkaline phosphatase or a polypeptide having alkaline phosphatase activity for the subject. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

Biomarkers/Endpoints for Diagnosis and/or Treatment of Muscle Weakness Diseases

A muscle weakness disease (such as HPP including, e.g., perinatal HPP, infantile HPP, childhood HPP, and odontohypophosphatasia, an HPP-like disease, CPPD, and familial hypophosphatemia, as described herein) can be treated with an alkaline phosphatase, or a polypeptide having alkaline phosphatase activity, (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa). The methods described herein are also useful for diagnosing a subject having or being prone to a muscle weakness disease, identifying a subject as having or being prone to a muscle weakness disease, or testing the efficacy of treatment of a muscle weakness disease. For example, a subject may be diagnosed as having or being prone to a muscle weakness disease if the subject is characterized as having certain characteristic biomarkers or metric scores. A subject may be treated with an alkaline phosphatase, or a polypeptide having alkaline phosphatase activity, (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), and the treatment efficacy or effects may be analyzed using the characteristic biomarkers or metric scores. Such biomarkers may include, e.g., the elevated inorganic pyrophosphate (PPi) concentration and/or the decreased alkaline phosphatase (ALP) in the serum, the bone or muscle tissues, or the urine of the subject. Exemplary metrics useful in the methods described herein for determining muscle weakness treatment efficacy may include: (1) the Bruininks-Oseretsky Test of Motor Proficiency 2nd Edition (BOT-2), (2) the Childhood Health Assessment Questionnaire (CHAQ), (3) the Pediatric Outcomes Data Collection Instrument (PODCI), (4) Bayley Scales of Infant and Toddler Development, 3^{rd} Edition (BSID-III), (5) the Peabody Developmental Motor Scales, 2nd Edition (PDMS-2), (6) the Six Minute Walk Test (6MWT), (7) the Muscle Strength Grade, and (8) Handheld Dynamometry (HHD), which are described in further detail below.

### Plasma Inorganic Pyrophosphate (PPi) and Alkaline Phosphatase (ALP) Concentrations

A subject having or being prone to a muscle weakness disease can be identified for treatment with an alkaline phosphatase or a polypeptide having alkaline phosphatase activity, (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) by determining the inorganic pyrophosphate (PPi) and/or alkaline phosphatase (ALP) concentrations in a sample, such as a plasma or urine sample, from the subject. Any method known to those of skill in the art can be used to quantify the PPi and/or ALP concentrations in a plasma sample or alternatively in a urine sample, as described in detail in Whyte et al., 1995 (J. Clin. Invest. 95(4): 1440-1445), hereby incorporated by reference in its entirety. Methods to quantify PPi concentrations in a plasma or urine sample are also described in Cheung et al., 1977 (Anal. Biochem. 83:61-63), Cook et al., 1978 (Anal. Biochem. 91:557-565), and Johnson et al, 1968 (Anal. Biochem. 26:137-145), which are each hereby incorporated by reference in their entirety.

In particular, an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) can be administered to a subject (e.g., a subject without a loss-of-function mutation in ALPL) having or being prone to a muscle weakness disease previously determined to have a plasma PPi concentration of up to about 6 μM (e.g., about 4.5 μM, about 5 μM, or about 5.5 μM or a plasma PPi concentration within the range of about 4.5 μM to about 6 μM). For example, the alkaline phosphatase or the polypeptide having alkaline phosphatase activity is administered to, e.g., an infant or child (e.g., a subject less than about 12 years of age) having a plasma PPi concentration of about 5.71 μM or greater; an adolescent (e.g., a subject of about 13 to about 18 years of age) having a plasma PPi concentration of about 4.78 μM or greater; or an adult (e.g., a subject of greater than about 18 years of age) having a plasma PPi concentration of about 5.82 μM or greater. Additionally, an alkaline phosphatase or a polypeptide having alkaline phosphatase activity can be administered to a subject (e.g., a human) having or being prone to a muscle weakness disease previously determined to have a plasma ALP concentration of, e.g., about 90 U/L or less for a subject of 0 to 14 days of age; about 134 U/L or less for a subject of 15 days of age to less than 1 year of age; about 156 U/L or less for a subject of about 1 year of age to less than 10 years of age; about 141 U/L or less for a subject of about 10 years of age to less than about 13 years of age; about 62 U/L or less for a female subject of about 13 years of age to less than about 15 years of age; about 127 U/L or less for a male subject of about 13 years of age to less than about 15 years of age; about 54 U/L or less for a female subject of about 15 years of age to less than about 17 years of age; about 89 U/L or less for a male subject of about 15 years of age to less than about 17 years of age; about 48 U/L or less for a female subject of about 17 years of age or older; or about 59 U/L or less for a male subject of about 17 years of age or older.

The plasma PPi concentration and/or plasma ALP concentration of a subject (e.g., a human) having or being prone to a muscle weakness disease can be compared to the plasma PPi concentration and/or plasma ALP of a normal subject to determine a treatment effect in the subject administered an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa). In particular, the alkaline phosphatase or the polypeptide having alkaline phosphatase activity can be administered for a treatment period of least one year (e.g., at least two years, at least three years, at least four years, at least five years, at least six years, at least seven years, at least eight years, at least nine years, at least ten years, or longer than ten years, such as for the lifetime of the subject). Alternatively, the methods can include determining the plasma PPi concentration and/or plasma ALP concentration prior to administering the alkaline phosphatase or the polypeptide having alkaline phosphatase activity to assess an effect in the subject of treatment with the alkaline phosphatase or the polypeptide having alkaline phosphatase activity.

The methods result in a decrease in PPi and/or an increase in ALP concentration in a sample (e.g., a plasma sample) from a subject (e.g., a human subject, e.g., a human subject without a loss-of-function mutation in ALPL) having or being prone to a muscle weakness disease. For example, treatment with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) results in a decrease in PPi concentration in a sample (e.g., a plasma sample) from the subject of about 1 μM, about 1.5 μM, about 2 μM, about 2.5 μM, or about 3 μM or 25% or greater (e.g., 30%, 35%, 40%, 45%, 50%, 55%, 60%, or more than 60%). Thus, the subject exhibits a plasma PPi concentration of, e.g., about 2 μM to about 5 μM, about 3 μM to about 5 μM, about 2 μM to about 4 μM, or about 2 μM to about 3 μM after administration of the alkaline phosphatase or the polypeptide having alkaline phosphatase activity.

Likewise, treatment with alkaline phosphatase or a polypeptide having alkaline phosphatase activity results in an increase in ALP concentration in a sample (e.g., a plasma sample) from a subject (e.g., a human) having or being prone to a muscle weakness disease of 30%, 35%, 40%, 45%, 50%, 55%, 60%, or more than 60%, relative to the subject prior to administration of the alkaline phosphatase or a polypeptide having alkaline phosphatase activity. For example, administration of the alkaline phosphatase or the polypeptide having alkaline phosphatase activity increases the ALP concentration in a sample (e.g., a plasma sample) from the subject to, e.g., about 273 U/L or greater for a subject of 0 to 14 days of age; about 518 U/L or greater for a subject of 15 days of age to less than 1 year of age; about 369 U/L or greater for a of about 1 year of age to less than 10 years of age; about 460 U/L or greater for a subject of about 10 years of age to less than about 13 years of age; about 280 U/L or greater for a female subject of about 13 years of age to less than about 15 years of age; about 517 U/L or greater for a male subject of about 13 years of age to less than about 15 years of age; about 128 U/L or greater for a female subject of about 15 years of age to less than about 17 years of age; about 365 U/L or greater for a male subject of about 15 years of age to less than about 17 years of age; about 95 U/L or greater for a female subject of about 17 years of age or older; or about 164 U/L or greater for a male subject of about 17 years of age or older.

The decrease in the plasma PPi and/or increase in the ALP concentrations of the subject (e.g., a human) having or being prone to a muscle weakness disease can be sustained throughout administration of the alkaline phosphatase or the polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa). For instance, the plasma PPi concentration decreases by about 25% and remains at +10% of the decreased plasma PPi concentration during treatment with the sALP and/or the plasma ALP concentration increases by about 50% and remains at +10% of the increased plasma ALP concentration during treatment with the alkaline phosphatase or the polypeptide having alkaline phosphatase activity.

Alternatively, when administration of an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) does not result in an average decrease in PPi concentrations in a plasma sample from the subject (e.g., a human) having or being prone to a muscle weakness disease by about 25% or greater, the dosage and/or frequency of sALP administration can be changed in order to determine an effective amount of the sALP for the subject. Likewise, when administration of an alkaline phosphatase or a polypeptide having alkaline phosphatase activity does not result in an average increase in ALP concentrations in a plasma sample from the subject by about 50% or greater, the dosage and/or frequency of alkaline phosphatase or a polypeptide having alkaline phosphatase activity administration can be changed in order to determine an effective amount of the alkaline phosphatase or a polypeptide having alkaline phosphatase activity for the subject. For instance, the dosage of the alkaline phosphatase or a polypeptide having alkaline phosphatase activity can be increased from, e.g., about 2.1 mg/kg/week or about 3.5 mg/kg/week to about 6 mg/kg/week or about 9 mg/kg/week.

Assays for Identifying a Subject with Reduced Alkaline Phosphatase (ALP) Concentration

Biochemical and diagnostic assays may be used to identify a subject with reduced ALP concentration, which may be caused by reduced transcription of ALPL, reduced translation of ALPL mRNA, elevated or reduced posttranslational modification of TNSALP, and/or reduced TNSALP enzymatic activity. The subject may have mutation(s) (e.g., single nucleotide polymorphisms (SNPs)) in their genomes, e.g., in regulatory regions such as promoters, upstream open reading frames (uORFs), downstream ORFS, 3′ or 5′ untranslated regions of ALPL or other genes that are either related (e.g., interact directly or indirectly with ALPL or TNSALP) or unrelated to ALPL or TNSALP. The subject may have a mutation in a coding region for a protein or enzyme that is not TNSALP. For example, another protein that interacts with TNSALP or directly or indirectly modulates the function, activity, and/or expression of TNSALP may have a mutation that imparts a downstream effect on TNSALP concentration. The genome of a subject with muscle weakness may be sequenced (e.g., with whole genome sequencing (WGS), intron sequencing, or exon sequencing) to identify relevant mutation(s) outside of the ALPL gene coding regions that contribute to or cause the muscle weakness symptoms described herein.

Assays for measuring transcription of ALPL, translation of ALPL mRNA, posttranslational modification of TNSALP, and/or reduced TNSALP enzymatic activity are known to one of skill in the art. For example, assays for measuring transcription include, for example, quantitative PCR (qPCR), RT-PCR, RNA-Seq, and next-generation sequencing (NGS). These assays may generate information regarding dysregulation of transcription of ALPL or other genes. Assays for measuring translation include, for example, Western Blot analysis, ELISA, Bradford Assay, and polysome profiling. These assays can be used to quantify protein levels. Assays for measuring posttranslational modifications (e.g., phosphorylation or glycosylation, e.g., N-linked or O-linked) of TNSALP include, for example, Western Blot analysis or ELISA assays, e.g., using antibodies that bind the posttranslationally modified TNSALP. Assays that measure TNSALP activity are well known in the art and are described e.g., in PCT Publication No. WO2005103263, which is hereby incorporated by reference in its entirety. Assays that measure TNSALP activity include fluorescence-based enzymatic assays and hydroxyapatite binding. Also, TNSALP localization assays can be used to reveal if TNSALP is present at the cell membrane. Furthermore, biochemical analysis that measures ALP serum substrate levels (e.g., PPi, PLP, and PEA) can be used as an output for measuring TNSALP activity.

### Bruininks-Oseretsky Test of Motor Proficiency 2nd Edition (BOT-2)

An exemplary Bruininks-Oseretsky Test of Motor Proficiency 2nd Edition (BOT-2) is described in Bruininks, R. H. (2005). Bruininks-Oseretsky Test of Motor Proficiency, (BOT-2), Minneapolis, MN: Pearson Assessment, hereby incorporated by reference in its entirety. In particular, the BOT-2 can be used to evaluate physical impairments and mobility restrictions in a subject having or being prone to a muscle weakness disease (e.g., HPP-like disease) to generate a BOT-2 score for the subject.

The BOT-2 includes a range of tests to evaluate physical impairments of a subject, which can be performed with, e.g., a kit including the tests. The BOT-2 provides composite BOT-2 scores in the following areas: strength, running speed and agility, fine motor precision, fine motor integration, manual dexterity, bilateral coordination, balance, and upper-limb coordination. For example, a subject having or being prone to a muscle weakness disease can perform sit-ups, v-ups, standing long jump, wall sit, and/or push-ups to determine the BOT-2 strength score. A subject having or being prone to a muscle weakness disease can step over a balance beam and/or perform a shuttle run, two-legged side hop, and/or one-legged side hop to determine the BOT-2 running speed and agility score. A subject having or being prone to a muscle weakness disease can cut out a circle and/or connect dots to determine the BOT-2 fine motor precision score. A subject having or being prone to a muscle weakness disease can copy a star and/or copy a square to determine the BOT-2 fine motor integration score. A subject having or being prone to a muscle weakness disease can transfer pennies, sort cards, and/or string blocks to determine the manual dexterity score. A subject having or being prone to a muscle weakness disease can tap his or her foot and finger and/or perform jumping jacks to determine the BOT-2 bilateral coordination score. A subject having or being prone to a muscle weakness disease can walk forward on a line and/or stand on one leg on a balance beam to determine the BOT-2 balance score. A subject having or being prone to a muscle weakness disease can throw a ball at a target and/or catch a tossed ball to determine the BOT-2 upper-limb coordination score.

A subject having or being prone to a muscle weakness disease (e.g., an HPP-like disease) could perform tests in one or more of described areas (strength, running speed and agility, fine motor precision, fine motor integration, manual dexterity, bilateral coordination, balance, and upper-limb coordination) to generate a BOT-2 score indicative of physical impairments in the subject. Within each BOT-2 area (strength, running speed and agility, fine motor precision, fine motor integration, manual dexterity, bilateral coordination, balance, and upper-limb coordination), such subject could perform one or more tests to determine the BOT-2 score of the subject, e.g., the subject could perform one or more of sit-ups, v-ups, standing long jump, wall sit, and push-ups to determine the BOT-2 strength score. Thus, only one test (e.g., one test selected from the group of sit-ups, v-ups, standing long jump, wall sit, and push-ups) can be performed to determine the BOT-2 score (e.g., a BOT-2 strength score) of a subject having or being prone to a muscle weakness disease (e.g., an HPP-like disease).

Each of the BOT-2 scores (strength, running speed and agility, fine motor precision, fine motor integration, manual dexterity, bilateral coordination, balance, and upper-limb coordination) of the subject having or being prone to a muscle weakness disease (e.g., an HPP-like disease) can be compared to the BOT-2 score of a subject without the muscle weakness disease (e.g., an HPP-like disease) to, e.g., determine a baseline comparison of the BOT-2 score. Each of the BOT-2 scores (e.g., strength, running speed and agility, fine motor precision, fine motor integration, manual dexterity, bilateral coordination, balance, and upper-limb coordination) of the subject having or being prone to a muscle weakness disease (e.g., an HPP-like disease) can be compared to the BOT-2 score of other subjects having or being prone to the muscle weakness disease (e.g., an HPP-like disease) to, e.g., provide a relative BOT-2 score for the subject.

BOT-2 scores (e.g., strength, running speed and agility, fine motor precision, fine motor integration, manual dexterity, bilateral coordination, balance, and upper-limb coordination scores) range from about 0 to equal to or less than about 25, in which a score of about 10 to about 20 is considered representative of healthy subject (e.g., subject without the muscle weakness disease (e.g., an HPP-like disease)). A subject with an average BOT-2 score (e.g., strength, running speed and agility, fine motor precision, fine motor integration, manual dexterity, bilateral coordination, balance, and upper-limb coordination scores) of less than about 10 can be treated with an alkaline phosphatase, or a polypeptide having alkaline phosphatase activity, e.g., sALP, such as TNALP, for example the sALP polypeptide of SEQ ID NO: 1 or a polypeptide variant having at least 95% sequence identity to the sequence of SEQ ID NO: 1, e.g., asfotase alfa.

For example, a subject having or being prone to a muscle weakness disease with a BOT-2 strength score of less than 10 (e.g., about 0, about 1, about 2, about 3, about 4, about 5, about 6, about 7, about 8, about 9, or about 10) can be treated with a sALP (such as TNALP, for example the sALP polypeptide of SEQ ID NO: 1 or a polypeptide variant having at least 95% sequence identity to the sequence of SEQ ID NO: 1, e.g., asfotase alfa) for a period of time, up to the lifetime of the subject. Likewise, a subject having or being prone to a muscle weakness disease with a BOT-2 running speed and agility score of less than 10 (e.g., about 0, about 1, about 2, about 3, about 4, about 5, about 6, about 7, about 8, about 9, or about 10) can then be treated with a sALP (such as TNALP, for example the sALP polypeptide of SEQ ID NO: 1 or a polypeptide variant having at least 95% sequence identity to the sequence of SEQ ID NO: 1, e.g., asfotase alfa) for a period of time, up to the lifetime of the subject.

The methods can result in an improvement in the BOT-2 score (e.g., strength, running speed and agility, fine motor precision, fine motor integration, manual dexterity, bilateral coordination, balance, and/or upper-limb coordination score) of a subject having or being prone to a muscle weakness disease (e.g., an HPP-like disease). For example, treatment with an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), such as treatment with a sALP for a period of time, can result in an average increase in the BOT-2 strength score to about 10 to about 20 (e.g. about 10, about 11, about 12, about 13, about 14, about 15, about 16, about 17, about 18, about 19, or about 20). Additionally, treatment with a sALP (such as TNALP, for example the sALP polypeptide of SEQ ID NO: 1 or a polypeptide variant having at least 95% sequence identity to the sequence of SEQ ID NO: 1, e.g., asfotase alfa) can result in an average increase in the BOT-2 running speed and agility score to about 5 to about 20 (e.g. about 5, about 6, about 7, about 8, about 9, about 10, about 11, about 12, about 13, about 14, about 15, about 16, about 17, about 18, about 19, or about 20).

The increase in the BOT-2 score (e.g., strength, running speed and agility, fine motor precision, fine motor integration, manual dexterity, bilateral coordination, balance, and/or upper-limb coordination score) can be sustained throughout administration of the alkaline phosphatase, or the polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa), e.g., for a period of time. Likewise, the decrease in physical impairments of muscles after administration of the alkaline phosphatase, or the polypeptide having alkaline phosphatase activity, can be sustained throughout administration of the alkaline phosphatase, or the polypeptide having alkaline phosphatase activity.

The BOT-2 scores (strength, running speed and agility, fine motor precision, fine motor integration, manual dexterity, bilateral coordination, balance, and upper-limb coordination scores) of a subject having or being prone to a muscle weakness disease (such as, HPP) can be used singly or in combination with other metrics for assessing treatment efficacy using an alkaline phosphatase, or a polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the alkaline phosphatase, or the polypeptide having alkaline phosphatase activity, is effective for treating muscle impairments associated with the muscle weakness disease. For example, when administration of a sALP to a subject having or being prone to a muscle weakness disease results in an average increase in the BOT-2 running speed and agility score to about 5 or greater than about 5, in which the subject previously had an average BOT-2 running speed and agility score of less than about 5, then the sALP is considered to be effective at, e.g., treating physical impairments associated with a muscle weakness disease.

Additionally, within each BOT-2 area (strength, running speed and agility, fine motor precision, fine motor integration, manual dexterity, bilateral coordination, balance, and upper-limb coordination), a subject having or being prone to a muscle weakness disease (e.g., an HPP-like disease, CPPD, familial hypophosphatemia described herein, etc.) could perform one or more tests to determine the BOT-2 score of the subject.

Alternatively, when administration of an alkaline phosphatase, or a polypeptide having alkaline phosphatase activity, such as a sALP, does not result in an average increase in the BOT-2 running speed and agility score to greater than about 5, the dosage and/or frequency of administration can be changed in order to determine the effective amount of the alkaline phosphatase, or the polypeptide having alkaline phosphatase activity, for the subject having or being prone to the muscle weakness disease (e.g., an HPP-like disease). For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

### Childhood Health Assessment Questionnaire (CHAQ)

The Childhood Health Assessment Questionnaire (CHAQ) can be administered to evaluate the health status of children having a muscle weakness disease (e.g., an HPP-like disease) to generate a CHAQ index score for the child, as is described in Bruce & Fries (J. Rheumatol. 30(1): 167-178, 2003) and Klepper (Arthritis & Rheumatism, 49: S5-S14, 2003), hereby incorporated by reference in their entirety. The CHAQ includes eight categories of questions for dressing/grooming, arising, eating, walking, hygiene, reach, grip, and activities, in which a parent or guardian records the amount of difficulty the child with the muscle weakness disease (e.g., an HPP-like disease) has in performing the respective activities. The range of scores within each category is from 0 to 3, in which a score of 0 indicates without any difficulty; a score of 1 indicates with some difficulty; a score of 2 indicates with much difficulty; and a score of 3 indicates that the child is unable to perform the activity.

Children having or being prone to a muscle weakness disease with an average CHAQ index score (e.g., indicative of disability in activities of daily living (ADL) and/or pain) greater than about 0.8 (e.g., about 0.8, about 1, about 1.2, about 1.4, about 1.6, about 1.8, about 2.0, about 2.2, about 2.4, about 2.6, about 2.8, or about 3.0) can be treated by administering an alkaline phosphatase, or a polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa). For example, children with an average CHAQ index score of greater than about 0.8 can be treated by administering an alkaline phosphatase, or a polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa) for a period of time, up to the lifetime of the subject. Furthermore, a child having or being prone to a muscle weakness disease disclosed herein could be asked one or more questions in one or more of the eight categories (dressing/grooming, arising, eating, walking, hygiene, reach, grip, and activities) to arrive at an average CHAQ index score, and if the average CHAQ index score is greater than about 0.8, the child can be treated by administering an alkaline phosphatase, or a polypeptide having alkaline phosphatase activity, such as a sALP.

The CHAQ index score of a child having or being prone to a muscle weakness disease disclosed herein can be compared to the CHAQ index score of children without such muscle weakness disease to, e.g., determine the standard deviation of the CHAQ index score. Additionally, the CHAQ index score of a child having or being prone to a muscle weakness disease disclosed herein can be compared to the CHAQ index score of other children having or being prone to the muscle weakness disease disclosed herein to, e.g., determine the standard deviation of the CHAQ index score.

The methods can result in an improvement in the CHAQ index score (e.g., indicative of disability in ADL and/or pain) of the child having or being prone to a muscle weakness disease disclosed herein. For example, treatment with a sALP (such as TNALP, for example the sALP polypeptide of SEQ ID NO: 1 or a polypeptide variant having at least 95% sequence identity to the sequence of SEQ ID NO: 1, e.g., asfotase alfa), such as treatment with a sALP for a period of time, up to the lifetime of the child, can result in an average decrease in the CHAQ index score to about 0 to equal to or less than about 0.5 (e.g. about 0, about 0.1, about 0.2, about 0.4, or about 0.5) in children with an HPP-like disease.

The decrease in the CHAQ index score of the child having or being prone to a muscle weakness disease (e.g., an HPP-like disease) can be sustained throughout administration of the alkaline phosphatase, or the polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa), e.g., for a period of time, up to the lifetime of the child. Likewise, the increase in ADL and/or decrease in pain of the child can be sustained throughout administration of the sALP (such as TNALP, for example the sALP polypeptide of SEQ ID NO: 1 or a polypeptide variant having at least 95% sequence identity to the sequence of SEQ ID NO: 1, e.g., asfotase alfa), for a period of time, up to the lifetime of the child.

The CHAQ index score of a child having or being prone to a muscle weakness disease (e.g., an HPP-like disease) can be used to assess treatment efficacy using an alkaline phosphatase, or a polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the alkaline phosphatase, or the polypeptide having alkaline phosphatase activity, is effective for treating, e.g., disability in activities of daily living (ADL) and pain associated with the muscle weakness disease. In particular, a child having or being prone to a muscle weakness disease could be asked one or more questions in one or more of the eight categories (dressing/grooming, arising, eating, walking, hygiene, reach, grip, and activities) to arrive at an average CHAQ index score and to assess treatment efficacy of sALP administration. For example, when administration of a sALP to a child having or being prone to a muscle weakness disease results in an average decrease in the CHAQ index score to equal to or less than about 0.5, in which the child previously had an average CHAQ index score of greater than about 0.8, then the sALP is effective at treating, e.g., disability in activities of daily living (ADL) and pain associated with a muscle weakness disease. Alternatively, when administration of a sALP does not result in an average decrease in the CHAQ index score to equal to or less than about 0.5, the dosage and/or frequency of sALP administration can be changed in order to determine the effective amount of the sALP for the child having or being prone to a muscle weakness disease. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

### Pediatric Outcomes Data Collection Instrument (PODCI)

Certain subjects having or being prone to a muscle weakness disease (e.g., an HPP-like disease) can be identified for treatment with an alkaline phosphatase, or a polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa) using the Pediatric Outcomes Data Collection Instrument (PODCI). The PODCI can be administered to evaluate the health status of children to generate a PODCI score for the subject, as is described in Plint et al. (J. Pediatr. Orthop. 23(6): 788-790, 2003). The PODCI includes eight categories of questions that can be completed by a subject having or being prone to a muscle weakness disease (e.g., an HPP-like disease) or by a parent/guardian of the subject. Categories that can be used to determine the PODCI of a subject having or being prone to a muscle weakness disease include the following: 1) the upper extremity and physical function scale to measure difficulty encountered in performing daily personal care and student activities; 2) the transfer and basic mobility scale to measure difficulty experienced in performing routine motion and motor activities in daily activities; 3) the sports/physical functioning scale to measure difficulty or limitations encountered in participating in more active activities or sports; 4) the pain/comfort scale to measure the level of pain experienced during the past week; 5) the treatment expectations scale to measure the long term expectations of treatment; 6) the happiness scale to measure overall satisfaction with personal looks and sense of similarity to friends and others of own age; 7) the satisfaction with symptoms scale to measure the subject's acceptance of current limitations should this be a life-long state; and 8) the global functioning scale, which is a general combined scale calculated from the first four scales listed above. In each of the categories, a standardized score is determined for the subject having or being prone to a muscle weakness disease and then converted to a 0 to 100 scale, in which 0 represents significant disability and 100 represents less disability.

A subject having or being prone to a muscle weakness disease (e.g., an HPP-like disease) with an average PODCI score (e.g., indicative of disability in ADL and/or pain) less than about 40 (e.g., about 5, about 10, about 15, about 20, about 25, about 30, about 35, or about 39) can be treated by administering an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa). For example, a subject with an average PODCI score of less than 40 can be treated by administering a sALP for a period of time, up to the lifetime of the subject. Furthermore, a subject having or being prone to a muscle weakness disease could be asked one or more questions in one or more of the eight scales described above (e.g., transfer and basic mobility, sports/physical functioning, and the pain/comfort scale) to arrive at an average PODCI score, and if the average PODCI score is greater than less than 40, the subject can be treated by administering a sALP.

The methods described herein can result in an increase in the PODCI score (e.g., indicative of disability in ADL and/or pain) of the subject having or being prone to a muscle weakness disease. For example, treatment with an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), such as treatment with a sALP for a period of time, up to the lifetime of the subject, can result in an average increase in the PODCI score to about 40 to about 50 (e.g. about 40, about 41, about 42, about 43, about 44, about 45, about 46, about 47, about 48, about 49, or about 50).

The increase in the PODCI score can be sustained throughout administration of the alkaline phosphatase, or the polypeptide having alkaline phosphatase activity, such as the sALP (e.g., TNALP, for example the sALP polypeptide of SEQ ID NO: 1 or a polypeptide variant having at least 95% sequence identity to the sequence of SEQ ID NO: 1, e.g., asfotase alfa), e.g., for a period of time, up to the lifetime of the subject having or being prone to a muscle weakness disease. Likewise, the increase in ADL and/or decrease in pain can be sustained throughout administration of the sALP (such as TNALP, for example the sALP polypeptide of SEQ ID NO: 1 or a polypeptide variant having at least 95% sequence identity to the sequence of SEQ ID NO: 1, e.g., asfotase alfa), for a period of time, up to the lifetime of the subject.

The PODCI score of a subject having or being prone to a muscle weakness disease (e.g., an HPP-like disease) can be used to assess treatment efficacy using an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the alkaline phosphatase, or the polypeptide having alkaline phosphatase activity, is effective for treating, e.g., disability in activities of daily living (ADL) and pain associated with the muscle weakness disease. In particular, a subject having or being prone to a muscle weakness disease could be asked one or more questions in one or more of the eight scales (the upper extremity and physical function scale, the transfer and basic mobility scale, the sports/physical functioning scale, the pain/comfort scale, the treatment expectations scale, the happiness scale, the satisfaction with symptoms scale, and the global functioning scale) to arrive at an average PODCI score and to assess treatment efficacy of sALP administration.

For example, when administration of a sALP to a subject having or being prone to a muscle weakness disease results in an average increase in the PODCI score to about 40 or greater than about 40, in which the subject previously had an average PODCI score of less than about 40, then the sALP is effective at treating, e.g., disability in activities of daily living (ADL) and pain associated with a muscle weakness disease. Alternatively, when administration of a sALP does not result in an average increase in the PODCI score to about 40 or greater than about 40, the dosage and frequency of sALP administration can be changed in order to determine the effective amount of the sALP for the subject having or being prone to a muscle weakness disease. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

### Bayley Scales of Infant and Toddler Development, 3^{rd} Edition (BSID-III)

Another endpoint, the Bayley Scales of Infant and Toddler Development, 3^{rd} Edition (BSID-III) can be administered to evaluate the health status of a subject having or being prone to a muscle weakness disease (e.g., an HPP-like disease) from birth to generate a BSID-III score for the subject, as is described in Bayley. (2006). Bayley scales of infant and toddler development: administration manual. San Antonio, TX: Harcourt Assessment. The BSID-III includes a series of developmental play tasks that can be administered to the subject to determine the raw BSID-III score. For example, categories for determining the BSID-III score of a subject having or being prone to a muscle weakness disease (e.g., infants of about three years of age or less having HPP) can include prehension, perceptual-motor integration, motor planning and speed, visual tracking, reaching, object grasping, object manipulation, functional hand skills, responses to tactile information, movement of the limbs and torso, static positioning, dynamic movement, balance, and motor planning. The BSID-III measurements are then converted to scaled BSID-III scores, which can be used to determine the subject's performance compared to healthy, age-adjusted subjects. The BSID-III scaled score of a subject having or being prone to a muscle weakness disease (e.g., a subject with an HPP-like disease) can range from 0 to 14, in which scores of about 7 to about 13 are considered the normal range for a healthy subject.

A subject having or being prone to a muscle weakness disease could perform tests in one or more of described categories (prehension, perceptual-motor integration, motor planning and speed, visual tracking, reaching, object grasping, object manipulation, functional hand skills, responses to tactile information, movement of the limbs and torso, static positioning, dynamic movement, balance, and motor planning) as an infant (e.g., at about 3 years of age or less than 3 years of age) to generate a BSID-III score indicative of delayed motor development. A subject having or being prone to a muscle weakness disease with an average BSID-III score in one or more of the described categories (prehension, perceptual-motor integration, motor planning and speed, visual tracking, reaching, object grasping, object manipulation, functional hand skills, responses to tactile information, movement of the limbs and torso, static positioning, dynamic movement, balance, and motor planning) less than about 2 as an infant can be treated by administering a sALP (such as TNALP, for example the sALP polypeptide of SEQ ID NO: 1 or a polypeptide variant having at least 95% sequence identity to the sequence of SEQ ID NO: 1, e.g., asfotase alfa). In particular, a subject having or being prone to a muscle weakness disease with an average BSID-III score of less than about 2 as an infant can be treated by administering a sALP for a period of time, up to the lifetime of the subject.

The methods can result in an improvement in the average BSID-III score (e.g., indicative of delayed motor development) of the subject having or being prone to a muscle weakness disease. For example, treatment with a sALP (such as TNALP, for example the sALP polypeptide of SEQ ID NO: 1 or a polypeptide variant having at least 95% sequence identity to the sequence of SEQ ID NO: 1, e.g., asfotase alfa), such as treatment with a sALP for a period of time, up to the lifetime of the subject, can result in an average increase in the BSID-III score to greater than about 5 (e.g., about 5, about 6, about 7, about 8, about 9, about 10, about 11, about 12, or about 13).

The increase in the BSID-III score can be sustained throughout administration of the alkaline phosphatase, or the polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), for a period of time, up to the lifetime of the subject having or being prone to a muscle weakness disease. Likewise, the increase in motor development can be sustained throughout administration of the alkaline phosphatase, or the polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), e.g., for a period of time, up to the lifetime of the subject.

The BSID-III score of a subject having or being prone to a muscle weakness disease (e.g., an HPP-like disease) can be used to assess treatment efficacy using an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the alkaline phosphatase, or the polypeptide having alkaline phosphatase activity, is effective for treating, e.g., delayed motor development associated with the muscle weakness disease. In particular, a subject having or being prone to a muscle weakness disease could perform tests in one or more of described categories (prehension, perceptual-motor integration, motor planning and speed, visual tracking, reaching, object grasping, object manipulation, functional hand skills, responses to tactile information, movement of the limbs and torso, static positioning, dynamic movement, balance, and motor planning) as an infant (e.g., at about three years of age or less having HPP) to arrive at an average BSID-III score and to assess treatment efficacy of sALP administration.

For example, when administration of a sALP to a child having or being prone to a muscle weakness disease results in an average increase in the BSID-III scaled score to greater than about 5, in which the child previously had an average BSID-III scaled score of less than about 2 as an infant (e.g., at about 3 years of age or less than 3 years of age), then the sALP is effective at treating, e.g., delayed motor development associated with an HPP-like disease. Alternatively, when administration of a sALP does not result in an average increase in the BSID-III scaled score to greater than about 5, the dosage and/or frequency of sALP administration can be changed in order to determine the effective amount of the sALP for the child having or being prone to a muscle weakness disease. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

### Peabody Developmental Motor Scales, 2nd Edition (PDMS-2)

Another endpoints, the Peabody Developmental Motor Scales, 2nd Edition (PDMS-2), can be administered to evaluate the health status of a subject having or being prone to a muscle weakness disease (e.g., an HPP-like disease) from birth to generate a PDMS-2 score for the subject, as is described in van Hartingsveldt et al. (Occup. Ther. Int. 12(1): 1-13, 2005). The PDMS-2 includes six categories of subtests to measure motor skills of the subject, such as a subject having HPP

In particular, PDMS-2 measurements can be determined from the following subtests: 1) the locomotor subtest to measure a subject's ability to move from one place to another (measurements include crawling, walking, running, hopping, and jumping forward); 2) the reflexes subtest to measure a subject's ability to automatically react to environmental events; 3) the stationary subtest to measure a subject's ability to sustain body control within the center of gravity and retain equilibrium; 4) the object manipulation subtest to measure a subject's ability to manipulate an object, such as catching, throwing, and kicking a ball; 5) the grasping subtest to measure a subject's ability to use his or her hands, such as the ability to hold an object with one hand and actions involving the controlled use of the fingers of both hands; and 6) the visual-motor integration subtest to measure a subject's ability to use his or her visual perceptual skills to perform complex eye-hand coordination tasks, such as reaching and grasping for an object, building with blocks, and copying designs. The PDMS-2 measurement can be determined for one or more of these categories for a subject having or being prone to a muscle weakness disease (e.g., an HPP-like disease) and then converted into a PDMS-2 score, such as the PDMS-2 locomotor standard score ranging from 0 to 13, in which the range of healthy subjects (e.g., subjects without the muscle weakness disease) is from about 7 to about 13.

A subject having or being prone to a muscle weakness disease with an average PDMS-score (e.g., indicative of delayed motor development) can be treated by administering a sALP (such as TNALP, for example the sALP polypeptide of SEQ ID NO: 1 or a polypeptide variant having at least 95% sequence identity to the sequence of SEQ ID NO: 1, e.g., asfotase alfa).

The methods described herein can result in an improvement in the PDMS-2 score (e.g., indicative of delayed motor development) of the subject having or being prone to a muscle weakness disease. For example, treatment with an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), can result in an average increase in the PDMS-2 score to about 7 to about 13 (e.g., about 7, about 8, about 9, about 10, about 11, about 12, or about 13).

The increase in the PDMS-2 score can be sustained throughout administration of the alkaline phosphatase, or the polypeptide having alkaline phosphatase activity, such as sALP (e.g., TNALP, for example the sALP polypeptide of SEQ ID NO: 1 or a polypeptide variant having at least 95% sequence identity to the sequence of SEQ ID NO: 1, e.g., asfotase alfa), for an elongated time, e.g., for a period of time, up to the lifetime of the subject having or being prone to a muscle weakness disease. Likewise, the increase in motor development can be sustained throughout administration of the sALP (such as TNALP, for example the sALP polypeptide of SEQ ID NO: 1 or a polypeptide variant having at least 95% sequence identity to the sequence of SEQ ID NO: 1, e.g., asfotase alfa) for a period of time, up to the lifetime of the subject having or being prone to a muscle weakness disease.

The PDMS-2 score of a subject having or being prone to a muscle weakness disease (e.g., an HPP-like disease) can be used to assess treatment efficacy using an alkaline phosphatase, or a polypeptide having alkaline phosphatase activity, such as a sALP (e.g., TNALP, for example the sALP polypeptide of SEQ ID NO: 1 or a polypeptide variant having at least 95% sequence identity to the sequence of SEQ ID NO: 1, e.g., asfotase alfa), in which improvements relative to a certain test score demonstrate that the alkaline phosphatase, or the polypeptide having alkaline phosphatase activity, is effective for treating, e.g., delayed motor development associated with the muscle weakness disease. For example, a child having or being prone to a muscle weakness disease could perform tests in one or more of described categories (locomotor, reflexes, stationary, object manipulation, grasping, and visual-motor) at about 5 years of age or less than 5 years of age to arrive at an average PDMS-2 score and to assess treatment efficacy of sALP administration.

For example, when administration of a sALP to a child having or being prone to a muscle weakness disease results in an average increase in the PDMS-2 standard score to about 7, in which the child previously had an average PDMS-2 standard score of about 5, then the sALP is effective at treating, e.g., delayed motor development associated with an HPP-like disease. Alternatively, when administration of a sALP does not result in an average increase in the PDMS-2 standard score to about 7, the dosage and/or frequency of sALP administration can be changed in order to determine the effective amount of the sALP for the child. For instance, the dosage of the sALP (such as TNALP, for example the sALP polypeptide of SEQ ID NO: 1 or a polypeptide variant having at least 95% sequence identity to the sequence of SEQ ID NO: 1, e.g., asfotase alfa) can be increased from, e.g., about 3 mg/kg/week to about 6 mg/kg/week or about 6 mg/kg/week to about 9 mg/kg/wk.

### Six Minute Walk Test (6MWT)

A subject having a muscle weakness disease can be identified for treatment with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) using the 6MWT. In particular, the 6MWT can be used to evaluate walking ability in an adult having a muscle weakness disease to generate a 6MWT value for the adult. The 6MWT can be performed indoors or outdoors using a flat, straight, enclosed corridor (e.g., of about 30 meters in length) with a hard surface. A stopwatch or other timer can be used to track the time and a mechanical counter or other device can be used to determine the distance (e.g., in meters) that the subject having a muscle weakness disease walks. For instance, the length of the corridor can be marked every three meters to determine the number of meters walked by the subject having a muscle weakness disease, with the turnaround point at 30 meters and the starting line also marked. The distance walked by the subject having a muscle weakness disease in six minutes can then be compared to the predicted number of meters walked, e.g., by a normal subject of about the same age, the same gender, and/or the same height, and expressed as a percentage value to generate the 6MWT value of the subject. The 6MWT value of the subject having a muscle weakness disease can be compared to the 6MWT value at baseline of the subject. Additionally, the 6MWT value of the subject having a muscle weakness disease can be compared to the 6MWT value of a normal subject.

A subject having a muscle weakness disease with an average 6MWT of less than about 80% of the predicted 6MWT value (e.g., relative to a normal subject of about the same age, the same gender, and/or the same height) can be treated with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), such as by administering an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks). For example, a subject having a muscle weakness disease with an average 6MWT of less than about 80% of the predicted 6MWT value (e.g., about 50%, about 55%, about 60%, about 65%, about 70%, or about 75% of the predicted 6MWT value) can be treated with an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks).

The methods can result in an improvement in the 6MWT value of a subject having a muscle weakness disease. For example, treatment with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), such as treatment with an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks), can result in an average increase in the 6MWT value to about 80% or greater of the predicted 6MWT value of the subject (e.g. about 82%, about 84%, about 86%, about 88%, about 90%, about 92%, about 94%, about 96%, about 98%, or more of the predictive 6MWT value).

The increase in the 6MWT value of the subject having a muscle weakness disease can be sustained throughout administration of the alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks). For instance, the 6MWT value increases to greater than about 80% of the predicted 6MWT value of the subject having a muscle weakness disease and remains at +10% of the increased 6MWT value during treatment with the alkaline phosphatase or a polypeptide having alkaline phosphatase activity.

Likewise, the improvement in walking ability of the subject having a muscle weakness disease can be sustained throughout administration of the alkaline phosphatase or a polypeptide having alkaline phosphatase activity, e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks). For instance, the subject having a muscle weakness disease exhibits decreased reliance on an assistive mobility device, such as a walker, a wheelchair, braces, crutches, or orthotics, during treatment with the sALP.

Alternatively, when administration of an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) does not result in an average increase in the 6MWT value to greater than 80% of the predicted 6MWT value (e.g., of a normal subject of about the same age, same gender, and/or height), the dosage and/or frequency of alkaline phosphatase or a polypeptide having alkaline phosphatase activity administration can be changed in order to determine the effective amount of the alkaline phosphatase or a polypeptide having alkaline phosphatase activity for the subject having a muscle weakness disease. For instance, the dosage of the alkaline phosphatase or a polypeptide having alkaline phosphatase activity can be increased from, e.g., about 2.1 mg/kg/week or about 3.5 mg/kg/week to about 6 mg/kg/week or about 9 mg/kg/week.

### Handheld Dynamometry (HHD)

The grip and muscle strength of a subject having or being prone to a muscle weakness disease can be assessed using Hand Held Dynamometry (HHD). For example, knee flexion and extension and also hip flexion, extension, and abduction of a subject having or being prone to a muscle weakness disease can be measured using, e.g., a MICROFET2™ Dynamometer, while grip strength of the subject can be measured using, e.g., a Jamar Grip Dynamometer. In particular, the administrator holds the dynamometer stationary, and the subject exerts a maximal force against the dynamometer. Peak force data is collected in pounds, then converted to Newtons (N). Torque values are then calculated using limb length in N-meters. The torque value can then be compared to the torque value of, e.g., a normal subject of about the same age, the same gender, and/or the same height, and expressed as a percentage value to generate the HHD value of the subject.

A subject having a muscle weakness disease with an average HHD value of less than about 80% of the predicted HHD value (e.g., relative to a normal subject of about the same age, the same gender, and/or the same height) can be treated with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), such as by administering an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks). For example, a subject having a muscle weakness disease with an average HHD of less than about 80% of the predicted HHD value (e.g., about 30%, about 35%, about 40%, about 45%, about 50%, about 55%, about 60%, about 65%, about 70%, or about 75% of the predicted HHD value) can be treated with an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks).

The methods can result in an improvement in the HHD value of a subject having a muscle weakness disease. For example, treatment with an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), such as treatment with an alkaline phosphatase or a polypeptide having alkaline phosphatase activity for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks), can result in an average increase in the HHD value to about 80% or greater of the predicted HHD value of the subject (e.g., about 83%, about 85%, about 87%, about 90%, about 93%, about 95%, about 97%, or about 100%, or about 100% of the predictive HHD value).

The increase in the HHD value of the subject having a muscle weakness disease can be sustained throughout administration of the alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the subject; particularly at least six weeks). For instance, the HHD value increases to greater than about 80% of the predicted HHD value of the subject having a muscle weakness disease and remains at +10% of the increased HHD value during treatment with the alkaline phosphatase or a polypeptide having alkaline phosphatase activity.

Alternatively, when administration of an alkaline phosphatase or a polypeptide having alkaline phosphatase activity (e.g. a sALP, such as TNALP, for example the sALP polypeptide of SEQ ID NO: 1 or a polypeptide variant having at least 95% sequence identity to the sequence of SEQ ID NO: 1, e.g., asfotase alfa) does not result in an average increase in the HHD value to greater than 80% of the predicted HHD value (e.g., of a subject having a muscle weakness disease of about the same age, same gender, and/or height), the dosage and/or frequency of alkaline phosphatase or a polypeptide having alkaline phosphatase activity administration can be changed in order to determine the effective amount of the alkaline phosphatase or a polypeptide having alkaline phosphatase activity for the subject having a muscle weakness disease. For instance, the dosage of the alkaline phosphatase or a polypeptide having alkaline phosphatase activity can be increased from, e.g., about 2.1 mg/kg/week or about 3.5 mg/kg/week to about 6 mg/kg/week or about 9 mg/kg/week.

### Alkaline Phosphatase

Asfotase alfa is a human TNALP (hTNALP; SEQ ID NO: 1) fusion protein formulated for the treatment of HPP. In particular, asfotase alfa (SEQ ID NO: 1) can be used effectively to treat hypophosphatasia (HPP), its symptoms, and physical impairments associated therewith in a subject having or being prone to a muscle weakness disease for an extended period of time (e.g., at least one day, at least one week, at least two weeks, at least three weeks, at least one month, at least three months, at least six months, at least one year, at least two years, at least three years, at least four years, at least five years, at least six years, at least seven years, at least eight years, at least nine years, at least ten years, or more than ten years (e.g., for the lifetime of the subject)).

Given the results described herein, the present disclosure is not limited to a particular alkaline phosphatase (ALP) or nucleic acid sequence encoding an ALP. Alkaline phosphatases encompass a group of enzymes that catalyze the cleavage of a phosphate moiety (e.g., hydrolysis of pyrophosphate, PPi). There are four known mammalian alkaline phosphatase (ALP) isozymes: tissue nonspecific alkaline phosphatase (TNALP; described further below), placental alkaline phosphatase (PLALP) (e.g., Accession Nos. P05187, NP_112603, and NP_001623), germ cell alkaline phosphatase (GALP) (e.g., Accession No. P10696), and intestinal alkaline phosphatase (IALP) (e.g., Accession Nos. P09923 and NP_001622). In addition to the exemplary ALPs discussed above, this disclosure also provides any polypeptide having the identical or similar catalytic site structure and/or enzymatic activity of ALP for treating a subject having or being prone to a muscle weakness disease. Bone delivery conjugates including sALP are further described in PCT publication Nos: WO 2005/103263 and WO 2008/138131.

TNALPs that can be used according to the methods described herein include, e.g., human TNALP (Accession Nos. NP_000469, AAI10910, AAH90861, AAH66116, AAH21289, and AAI26166); rhesus TNALP (Accession No. XP_01109717); rat TNALP (Accession No. NP_037191); dog TNALP (Accession No. AAF64516); pig TNALP (Accession No. AAN64273), mouse (Accession No. NP_031457), cow TNALP (Accession Nos. NP_789828, NP_776412, AAM 8209, and AAC33858), and cat TNALP (Accession No. NP_001036028). In particular, TNALP can be a recombinant human TNALP (e.g., SEQ ID NO: 1, asfotase alfa; see U.S. Pat. Nos. 7,763,712 and 7,960,529, incorporated herein by reference in their entirety) used for the treatment of a subject having or being prone to a muscle weakness disease. The TNALP can also be one that exhibits at least about 95% sequence identity to the polypeptide or nucleic acid sequence of the above-noted TNALPs.

### Soluble Alkaline Phosphatase

The ALPs of the present invention include soluble (e.g., extracellular or non-membrane-bound) forms of any of the alkaline phosphatases described herein. The sALP can be, for example, a soluble form of human tissue non-specific alkaline phosphatase (human TNALP (hTNALP)). The present disclosure is not limited to a particular sALP and can include any sALP polypeptide that is physiologically active toward, e.g., phosphoethanolamine (PEA), inorganic pyrophosphate (PPi), and pyridoxal 5′-phosphate (PLP). In particular, a sALP of the present invention is catalytically competent to improve skeletal mineralization in bone. The present disclosure further includes nucleic acids encoding the sALPs described herein that can be used to treat muscle weakness conditions described herein, including e.g., HPP-like disease, CPPD, familial hypophosphatemia (such as autosomal dominant hypophosphatemic rickets (ADHR), autosomal recessive hypophosphatemic rickets, X-linked hypophosphatemic rickets, X-linked hypophosphatemia (XLH), etc.), etc.

TNALP is a membrane-bound protein anchored by a glycolipid moiety at the C-terminal (Swiss-Prot, P05186). This glycolipid anchor (GPI) is added post-translationally after the removal of a hydrophobic C-terminal end, which serves both as a temporary membrane anchor and as a signal for the addition of the GPI. While the GPI anchor is located in the cell membrane, the remaining portions of TNALP are extracellular. In particular, TNALP (e.g., human TNALP (hTNALP)) can be engineered to replace the first amino acid of the hydrophobic C-terminal sequence (an alanine) with a stop codon, thereby producing an engineered hTNALP that contains all amino acid residues of the native anchored form of TNALP and lacks the GPI membrane anchor. One skilled in the art will appreciate that the position of the GPI membrane anchor will vary in different ALPs and can include, e.g., the last 10, 12, 14, 16, 18, 20, 21, 22, 23, 24, 25, 26, 27, 28, 29, 30, 32, 34, 36, 38, 40, 45, 50, or more amino acid residues on the C-terminus of the polypeptide. Recombinant sTNALP can include, e.g., amino acids 1 to 502 (18 to 502 when secreted), amino acids 1 to 501 (18 to 501 when secreted), amino acids 1 to 504 (18 to 504 when secreted), amino acids 1 to 505 (18-505 when secreted), or amino acids 1 to 502. Thus, the C-terminal end of the native ALP can be truncated by certain amino acids without affecting ALP activity.

In addition to the C-terminal GPI anchor, TNALP also has an N-terminal signal peptide sequence. The N-terminal signal peptide is present on the synthesized protein when it is synthesized, but cleaved from TNALP after translocation into the ER. The sALPs include both secreted (i.e., lacking the N-terminal signal) and non-secreted (i.e., having the N-terminal signal) forms thereof. One skilled in the art will appreciate that the position of the N-terminal signal peptide will vary in different alkaline phosphatases and can include, for example, the first 5, 8, 10, 11, 12, 13, 14, 15, 16, 17, 18, 19, 20, 21, 22, 23, 24, 25, 27, 30, or more amino acid residues on the N-terminus of the polypeptide. One of skill in the art can predict the position of a signal sequence cleavage site, e.g., by an appropriate computer algorithm such as that described in Bendtsen et al. (J. Mol. Biol. 340(4):783-795, 2004) and available on the Web at www.cbs.dtu.dk/services/SignalP/.

The present invention also includes sALP consensus sequences derived from the extracellular domain of ALP isozymes (e.g., TNALP, PALP, GCALP, IALP, etc.). Thus, similar to STNALP discussed above, the present disclosure also provides other soluble human ALP isozymes, i.e., without the peptide signal, preferably comprising the extracellular domain of the ALPs. The sALPs also include polypeptide sequences satisfying a consensus sequence derived from the ALP extracellular domain of human ALP isozymes and of mammalian TNALP orthologs (human, mouse, rat, cow, cat, and dog) or a consensus derived from the ALP extracellular domain of just mammalian TNALP orthologs (human, mouse, rat, cow, cat, and dog). The sALPs also include those which satisfy similar consensus sequences derived from various combinations of these TNALP orthologs or human ALP isozymes. Such consensus sequences are given, for example, in WO 2008/138131.

sALPs of the present invention can include not only the wild-type sequence of the sALPs described above, but any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to these alkaline phosphatases (e.g., SEQ ID NOs: 1-24; for example the sALP polypeptide of SEQ ID NO: 1 or a polypeptide variant having at least 95% sequence identity to the sequence of SEQ ID NO: 1, e.g., asfotase alfa). Examples of mutations that can be introduced into an ALP sequence are described in US Publication No. 2013/0323244, hereby incorporated by reference in its entirety. A sALP can optionally be glycosylated at any appropriate one or more amino acid residues. In addition, an sALP can have at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to any of the sALPs described herein (such as TNALP, for example the sALP polypeptide of SEQ ID NO: 1 or a polypeptide variant having at least 95% sequence identity to the sequence of SEQ ID NO: 1, e.g., asfotase alfa). A sALP can have 1, 2, 3, 4, 5, 6, 7, 8, 9, 10, or more additions, deletions, or substitutions relative to any of the sALPs described herein (such as TNALP, for example the sALP polypeptide of SEQ ID NO: 1 or a polypeptide variant having at least 95% sequence identity to the sequence of SEQ ID NO: 1, e.g., asfotase alfa).

### SALP Fusion Polypeptides

Any of the sALPs and linkers described herein can be combined in a sALP polypeptide, e.g., a sALP polypeptide of A-sALP-B, wherein each of A and B is absent or is an amino acid sequence of at least one amino acid (such as TNALP, for example the sALP polypeptide of SEQ ID NO: 1 or a polypeptide variant having at least 95% sequence identity to the sequence of SEQ ID NO: 1, e.g., asfotase alfa). When present, A and/or B can be any linker described herein. In some sALP polypeptides, A is absent, B is absent, or A and B are both absent. The sALP polypeptides can optionally include an Fc region to provide an sALP fusion polypeptide, as described herein. The sALP polypeptide can optionally include a bone-targeting moiety, as described herein. In some sALP polypeptides, a linker, e.g., a flexible linker, can be included between the bone-targeting moiety and the sALP, such as a dipeptide sequence (e.g., leucine-lysine or aspartic acid-isoleucine). Further exemplary Fc regions, linkers, and bone-targeting moieties are described below.

Any of the sALPs, linkers, and Fc regions described herein can be combined in a fusion polypeptide, e.g., a recombinant fusion polypeptide, which includes the structure Z-sALP-Y-spacer-X-Wₙ-V, Z-Wₙ-X-spacer-Y-sALP-V, Z-sALP-Y-Wₙ-X-spacer-V, and Z-Wₙ-X-sALP-Y-spacer-V (such as TNALP, for example the sALP polypeptide of SEQ ID NO: 1 or a polypeptide variant having at least 95% sequence identity to the sequence of SEQ ID NO: 1, e.g., asfotase alfa). In particular, the structure can be Z-sALP-Y-spacer-X-Wₙ-V or Z-Wₙ-X-spacer-Y-sALP-V. The sALP can be the full-length or functional fragments of ALPs, such as the soluble, extracellular domain of the ALP, as is described herein (e.g., TNALP, PALP, GCALP and IALP). Any one of X, Y, Z, and V and/or the spacer can be absent or an amino acid sequence of at least one amino acid. Wₙ can be a bone-targeting moiety, e.g., having a series of consecutive Asp or Glu residues, in which n=1 to 50, e.g., n=3-30, e.g., 5-15, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 36, 38, 39, 40, 41, 42, 43, 44, 45, 46, 47, 48, 49, or 50. The bone-targeting moiety, if present, can be positioned anywhere in the fusion polypeptide, e.g., at or near the N-terminal or C-terminal end, and/or in the linker region. For instance, the bone-targeting moiety is at the C-terminal end. sALP polypeptides and fusion polypeptides can also lack a bone-targeting moiety.

sALP fusion polypeptides can have the structure hTNALP-Fc-D₁₀. In particular, sALP fusion polypeptides can include an amino acid sequence of SEQ ID NO: 1 or a polypeptide variant having at least 95% sequence identity to the sequence of SEQ ID NO: 1, e.g., asfotase alfa.

sALP fusion polypeptides can have a negatively charged polypeptide including from one to fifty (e.g., 6-10, 10-15, 10-16, e.g., 6, 7, 8, 9, 10, 11, 12, 13, 14, 15, 16, 17, 19, 19, 20, 25, 30, 35, 40, 45, or 50) negatively charged amino acids, such as an aspartic acid or glutamic acid residues. The negatively charged peptide may consist or include at least one of D₁₀, D₁₆, E₁₀, and E₁₆.

Useful spacers include, but are not limited to, polypeptides comprising an Fc, and hydrophilic and flexible polypeptides able to alleviate the repulsive forces caused by the presence of the terminal highly negatively charged peptide (e.g., Wₙ). For example, a sALP can be a fusion polypeptide including an Fc region of an immunoglobulin at the N-terminal or C-terminal domain. An immunoglobulin molecule has a structure that is well known in the art. It includes two light chains (~23 kD each) and two heavy chains (~50-70 kD each) joined by inter-chain disulfide bonds. Immunoglobulins are readily cleaved proteolytically (e.g., by papain cleavage) into Fab (containing the light chain and the VH and CH1 domains of the heavy chain) and Fc (containing the CH2 and CH3 domains of the heavy chain, along with adjoining sequences). Useful Fc fragments as described herein include the Fc fragment of any immunoglobulin molecule, including IgG, IgM, IgA, IgD, or IgE, and their various subclasses (e.g., IgG-1, IgG-2, IgG-3, IgG-4, IgA-1, IgA-2), from any mammal (e.g., human). For instance, the Fc fragment is human IgG-1. The Fc fragments can include, for example, the CH2 and CH3 domains of the heavy chain and any portion of the hinge region. The Fc region can optionally be glycosylated at any appropriate one or more amino acid residues known to those skilled in the art. In particular, the Fc fragment of the fusion polypeptide has the amino acid sequence of SEQ ID NO: 20, or has at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 20. Engineered, e.g., non-naturally occurring, Fc regions can also be used (see, e.g., International Application Pub. No. WO2005/007809, which is hereby incorporated by reference). An Fc fragment as described herein can have 1, 2, 3, 4, 5, 6, 7, 8, 9, 10, 11, 12, 13, 14, 15, 16, 17, 18, 19, 20, 25, 30, 35, 40, 50, or more additions, deletions, or substitutions relative to any of the Fc fragments described herein.

The sALP fusion polypeptides described herein (such as TNALP, for example the sALP polypeptide of SEQ ID NO: 1 or a polypeptide variant having at least 95% sequence identity to the sequence of SEQ ID NO: 1, e.g., asfotase alfa) can include a peptide linker region between the Fc fragment. In addition, a peptide linker region can be included between the Fc fragment and the optional bone-targeting moiety. The linker region can be of any sequence and length that allows the sALP to remain biologically active, e.g., not sterically hindered. Exemplary linker lengths are between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. For instance, linkers include or consist of flexible portions, e.g., regions without significant fixed secondary or tertiary structure. Exemplary flexible linkers are glycine-rich linkers, e.g., containing at least 50%, 60%, 65%, 70%, 75%, 80%, 85%, 90%, 95%, or even 100% glycine residues. Linkers can also contain, e.g., serine residues. In some cases, the amino acid sequence of linkers consists only of glycine and serine residues. A linker can optionally be glycosylated at any appropriate one or more amino acid residues. Additionally, a linker as described herein can include any other sequence or moiety, attached covalently or non-covalently. The linker can also be absent, in which the Fc fragment and the sALP are fused together directly, with no intervening residues. Certain Fc-sALP or sALP-Fc fusion polypeptides can be viewed, according to the present disclosure, either as 1) having no linker, or as 2) having a linker which corresponds to a portion of the sALP. For example, Fc fused directly to hsTNALP (1-502) can be viewed, e.g., either as having no linker, in which the hsTNALP is amino acids 1-502, or as having a 17-amino acid linker, in which the hsTNALP (18-502).

The sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be associated into dimers or tetramers. For example, two sALP-Fc monomers can covalently be linked through two disulfide bonds located in the hinge regions of the Fc fragments. Additionally, the polypeptide or fusion polypeptide (e.g., a sALP polypeptide or fusion polypeptide) can be glycosylated or PEGylated.

### Production of Nucleic Acids and Polypeptides

The nucleic acids encoding sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be produced by any method known in the art. Typically, a nucleic acid encoding the desired fusion polypeptide is generated using molecular cloning methods, and is generally placed within a vector, such as a plasmid or virus. The vector is used to transform the nucleic acid into a host cell appropriate for the expression of the fusion polypeptide. Representative methods are disclosed, for example, in Maniatis et al. (Cold Springs Harbor Laboratory, 1989). Many cell types can be used as appropriate host cells, although mammalian cells are preferable because they are able to confer appropriate post-translational modifications. Host cells of the present invention can include, e.g., Chinese Hamster Ovary (CHO) cell, L cell, C127 cell, 3T3 cell, BHK cell, COS-7 cell or any other suitable host cell known in the art. For example, the host cell is a Chinese Hamster Ovary (CHO) cell (e.g., a CHO-DG44 cell).

The sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be produced under any conditions suitable to effect expression of the sALP polypeptide in the host cell. Such conditions include appropriate selection of a media prepared with components such as a buffer, bicarbonate and/or HEPES, ions like chloride, phosphate, calcium, sodium, potassium, magnesium, iron, carbon sources like simple sugars, amino acids, potentially lipids, nucleotides, vitamins and growth factors like insulin; regular commercially available media like alpha-MEM, DMEM, Ham's-F12, and IMDM supplemented with 2-4 mM L-glutamine and 5% Fetal bovine serum; regular commercially available animal protein free media like Hyclone™ SFM4CHO, Sigma CHO DHFR⁻, Cambrex POWER™ CHO CD supplemented with 2-4 mM L-glutamine. These media are desirably prepared without thymidine, hypoxanthine and L-glycine to maintain selective pressure, allowing stable protein-product expression.

### Pharmaceutical Compositions, Formulations, and Dosing

A composition of the disclosure containing a sALP or sALP fusion polypeptide (such as, e.g., TNALP, for example the sALP polypeptide of SEQ ID NO: 1 or a polypeptide variant having at least 95% sequence identity to the sequence of SEQ ID NO: 1, e.g., asfotase alfa) can be formulated by a variety of methods known in the art. As will be appreciated by the skilled artisan, the route and/or mode of administration may be used to dictate the formulation. The route of administration can depend on a variety of factors, such as the environment and therapeutic goals. In particular, the polypeptides and fusion polypeptides described herein can be formulation for administration by any route known in the art, e.g., subcutaneous (e.g., by subcutaneous injection), intravenously, orally, nasally, intramuscularly, sublingually, intrathecally, or intradermally. By way of example, pharmaceutical compositions can be in the form of a liquid, solution, suspension, pill, capsule, tablet, gelcap, powder, gel, ointment, cream, nebulae, mist, atomized vapor, aerosol, or phytosome.

### Formulations

The compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be formulated according to standard methods. Pharmaceutical formulation is a well-established art, and is further described in, e.g., Gennaro (2000) “Remington: The Science and Practice of Pharmacy,” 20th Edition, Lippincott, Williams & Wilkins (ISBN: 0683306472); Ansel et al. (1999) “Pharmaceutical Dosage Forms and Drug Delivery Systems,” 7th Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). For instance, a sALP composition (such as TNALP, for example the sALP polypeptide of SEQ ID NO: 1 or a polypeptide variant having at least 95% sequence identity to the sequence of SEQ ID NO: 1, e.g., asfotase alfa) can be formulated, for example, as a buffered solution at a suitable concentration and suitable for storage at 2-8° C. (e.g., 4° C.). A composition can also be formulated for storage at a temperature below 0° C. (e.g., −20° C. or −80° C.). A composition can further be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, the compositions described herein can be stable in storage for at least 1 year at 2-8° C. (e.g., 4° C.).

The compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application.

For example, compositions intended for systemic or local delivery can be in the form of injectable or infusible solutions. Accordingly, the compositions (such as TNALP, for example the sALP polypeptide of SEQ ID NO: 1 or a polypeptide variant having at least 95% sequence identity to the sequence of SEQ ID NO: 1, e.g., asfotase alfa) can be formulated for administration by a parenteral mode (e.g., subcutaneous, intravenous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, subcutaneous, intradermal, intravenous, intranasal, intraocular, pulmonary, intramuscular, intra-arterial, intrathecal, intracapsular, intraorbital, intracardiac, intradermal, intrapulmonary, intraperitoneal, transtracheal, subcuticular, intraarticular, subcapsular, subarachnoid, intraspinal, epidural, intracerebral, intracranial, intracarotid, and intrasternal injection and infusion.

The compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The compositions described herein can also be formulated in immunoliposome compositions. Such formulations can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

Compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can also be formulated with a carrier that will protect the composition (e.g., a sALP polypeptide or sALP fusion polypeptide) against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

A composition containing a sALP or sALP fusion polypeptide (such as TNALP, for example the sALP polypeptide of SEQ ID NO: 1 or a polypeptide variant having at least 95% sequence identity to the sequence of SEQ ID NO: 1, e.g., asfotase alfa) can be formulated as a solution for injection, which is a clear, colorless to slightly yellow, aqueous solution, pH 7.4. The sALP or sALP polypeptide (such as TNALP, for example the sALP polypeptide of SEQ ID NO: 1 or a polypeptide variant having at least 95% sequence identity to the sequence of SEQ ID NO: 1, e.g., asfotase alfa) may be formulated at a concentration of 12 mg/0.3 mL, 18 mg/0.45 mL, 28 mg/0.7 mL, 40 mg/l ml, or 80 mg/0.8 mL. The sALP or sALP polypeptide (e.g., formulated at a concentration of 12 mg/0.3 mL, 18 mg/0.45 mL, 28 mg/0.7 mL, 40 mg/l ml, or 80 mg/0.8 mL) may be formulated with a carrier, such as sodium chloride and/or sodium phosphate (e.g., ~150 mM NaCl and/or ~25 mM sodium phosphate, in particular at a pH of ~7.4). In particular, the composition can be formulated as a 40 mg/ml solution for injection, in which each ml of solution contains 40 mg of sALP or sALP polypeptide (e.g., each vial contains 0.3 ml solution and 12 mg of sALP (40 mg/ml), each vial contains 0.45 ml solution and 18 mg of sALP (40 mg/ml), each vial contains 0.7 ml solution and 28 mg of sALP (40 mg/ml), or each vial contains 1.0 ml solution and 40 mg of asfotase alfa (40 mg/ml)). A sALP or sALP polypeptide (such as TNALP, for example the sALP polypeptide of SEQ ID NO: 1 or a polypeptide variant having at least 95% sequence identity to the sequence of SEQ ID NO: 1, e.g., asfotase alfa) can be formulated as a solution for injection at a concentration of 100 mg/ml, in which each 1 ml of solution contains 100 mg of sALP or sALP polypeptide (e.g., each vial contains 0.8 ml solution and 80 mg of asfotase alfa (100 mg/ml)).

When compositions are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times.

Compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be formulated for administration to a subject or, if administered to a fetus, to a female carrying such fetus, along with intravenous gamma globulin therapy (IVIG), plasmapheresis, plasma replacement, or plasma exchange.

### Carriers/Vehicles

Preparations containing a sALP or sALP fusion polypeptide (such as TNALP, for example the sALP polypeptide of SEQ ID NO: 1 or a polypeptide variant having at least 95% sequence identity to the sequence of SEQ ID NO: 1, e.g., asfotase alfa) can be provided to a subject having or being prone to a muscle weakness disease, in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. For example, the pharmaceutically acceptable carrier can include sodium chloride and/or sodium phosphate, in which the composition includes, e.g., about 150 mM sodium chloride and/or about 25 mM sodium phosphate, pH 7.4.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

### Dosage

The sALP polypeptides (such as TNALP, for example the sALP polypeptide of SEQ ID NO: 1 or a polypeptide variant having at least 95% sequence identity to the sequence of SEQ ID NO: 1, e.g., asfotase alfa), and/or the fusion proteins of SEQ ID NO: 21 or SEQ ID NO: 22, or a variant having at least 95% sequence identity to the sequence of SEQ ID NO: 21 or SEQ ID NO: 22, described herein, can be administered to a subject having or being prone to a muscle weakness disease, in individual doses ranging, e.g., from 0.01 mg/kg to 500 mg/kg (e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, 0.5 mg/kg to 25 mg/kg, 1.0 mg/kg to 10 mg/kg, 1.5 mg/kg to 5 mg/kg, or 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 μg/kg).

Exemplary doses of a sALP include, e.g., 0.01, 0.05, 0.1, 0.5, 1, 2, 2.5, 5, 10, 20, 25, 50, 100, 125, 150, 200, 250, or 500 mg/kg; or 1, 2, 2.5, 5, 10, 20, 25, 50, 100, 125, 150, 200, 250, 500, 750, 900, or 1,000 μg/kg. For all dosages or ranges recited herein, the term “about” can be used to modify these dosages by +10% of the recited values or range endpoints. In particular, compositions (e.g., including sALP (such as TNALP, for example the sALP polypeptide of SEQ ID NO: 1 or a polypeptide variant having at least 95% sequence identity to the sequence of SEQ ID NO: 1, e.g., asfotase alfa)) in accordance with the present disclosure can be administered to a subject in doses ranging from about 0.001 mg/kg/day to about 500 mg/kg/day, about 0.01 mg/kg/day to about 100 mg/kg/day, or about 0.01 mg/kg/day to about 20 mg/kg/day. For example, the sALP compositions (such as TNALP, for example the sALP polypeptide of SEQ ID NO: 1 or a polypeptide variant having at least 95% sequence identity to the sequence of SEQ ID NO: 1, e.g., asfotase alfa) can be administered to a subject in a weekly dosage ranging, e.g., from about 0.5 mg/kg/week to about 140 mg/kg/week, e.g., about 0.8 mg/kg/week to about 50 mg/kg/week, or about 1 mg/kg/week to about 10 mg/kg/week (e.g., about 6 or about 9 mg/kg/week). In particular, the sALP (such as TNALP, for example the sALP polypeptide of SEQ ID NO: 1 or a polypeptide variant having at least 95% sequence identity to the sequence of SEQ ID NO: 1, e.g., asfotase alfa) can be administered at a dosage of 2 mg/kg three times a week (total dose 6 mg/kg/week), 1 mg/kg six times a week (total dose 6 mg/kg/week), 3 mg/kg three times a week (total dose 9 mg/kg/week), 0.5 mg/kg three times a week (total dose of 1.5 mg/kg/week), or 9.3 mg/kg three times a week (total dose 28 mg/kg/week). The dosage will be adapted by the clinician in accordance with conventional factors such as the extent of the disease and different parameters from the subject having or being prone to a muscle weakness disease.

Dosages of compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa), and/or the fusion proteins of the SEQ ID NO: 21 or SEQ ID NO: 22, or a variant having at least 95% sequence identity to the sequence of SEQ ID NO: 21 or SEQ ID NO: 22 can be provided in either a single or multiple dosage regimens. Doses can be administered, e.g., hourly, bi-hourly, daily, bi-daily, twice a week, three times a week, four times a week, five times a week, six times a week, weekly, biweekly, monthly, bimonthly, or yearly. Alternatively, doses can be administered, e.g., twice, three times, four times, five times, six times, seven times, eight times, nine times, 10 times, 11 times, or 12 times per day. In particular, the dosing regimen is once weekly. The duration of the dosing regimen can be, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s), week(s), or month(s), or even for the remaining lifespan of the subject having or being prone to a muscle weakness disease. The amount, frequency, and duration of dosage will be adapted by the clinician in accordance with conventional factors such as the extent of the disease and different parameters from the subject having or being prone to a muscle weakness disease.

For example, the recommended dosage of a sALP or sALP fusion polypeptide ((such as TNALP, for example the sALP polypeptide of SEQ ID NO: 1 or a polypeptide variant having at least 95% sequence identity to the sequence of SEQ ID NO: 1, e.g., asfotase alfa) is 2 mg/kg of body weight administered subcutaneously three times per week, or a dosage regimen of 1 mg/kg of body weight administered subcutaneously six times per week. Additional dosage information is provided below (Table 1).

TABLE 1
DOSING OF ASFOTASE ALFA

| | If injecting 3× per week | | | | | If injecting 6× per week | | | | |
|---|---|---|---|---|---|---|---|---|---|---|
| Body | | | Volume | Vial type | | | | Volume | Vial type | |
| Weight | Dose to be | | to be | used for | | Dose to be | | to be | used for | |
| (kg) | injected | | injected | injection | | injected | | injected | injection | |
| 3 | 6 | mg | 0.15 ml | 0.3 | ml | | | | | |
| 4 | 8 | mg | 0.20 ml | 0.3 | ml | | | | | |
| 5 | 10 | mg | 0.25 ml | 0.3 | ml | | | | | |
| 6 | 12 | mg | 0.30 ml | 0.3 | ml | 6 | mg | 0.15 ml | 0.3 | ml |
| 7 | 14 | mg | 0.35 ml | 0.45 | ml | 7 | mg | 0.18 ml | 0.3 | ml |
| 8 | 16 | mg | 0.40 ml | 0.45 | ml | 8 | mg | 0.20 ml | 0.3 | ml |
| 9 | 18 | mg | 0.45 ml | 0.45 | ml | 9 | mg | 0.23 ml | 0.3 | ml |
| 10 | 20 | mg | 0.50 ml | 0.7 | ml | 10 | mg | 0.25 ml | 0.3 | ml |
| 11 | 22 | mg | 0.55 ml | 0.7 | ml | 11 | mg | 0.28 ml | 0.3 | ml |
| 12 | 24 | mg | 0.60 ml | 0.7 | ml | 12 | mg | 0.30 ml | 0.3 | ml |
| 13 | 26 | mg | 0.65 ml | 0.7 | ml | 13 | mg | 0.33 ml | 0.45 | ml |
| 14 | 28 | mg | 0.70 ml | 0.7 | ml | 14 | mg | 0.35 ml | 0.45 | ml |
| 15 | 30 | mg | 0.75 ml | 1 | ml | 15 | mg | 0.38 ml | 0.45 | ml |
| 16 | 32 | mg | 0.80 ml | 1 | ml | 16 | mg | 0.40 ml | 0.45 | ml |
| 17 | 34 | mg | 0.85 ml | 1 | ml | 17 | mg | 0.43 ml | 0.45 | ml |
| 18 | 36 | mg | 0.90 ml | 1 | ml | 18 | mg | 0.45 ml | 0.45 | ml |
| 19 | 38 | mg | 0.95 ml | 1 | ml | 19 | mg | 0.48 ml | 0.7 | ml |
| 20 | 40 | mg | 1.00 ml | 1 | ml | 20 | mg | 0.50 ml | 0.7 | ml |
| 25 | 50 | mg | 0.50 ml | 0.8 | ml | 25 | mg | 0.63 ml | 0.7 | ml |
| 30 | 60 | mg | 0.60 ml | 0.8 | ml | 30 | mg | 0.75 ml | 1 | ml |
| 35 | 70 | mg | 0.70 ml | 0.8 | ml | 35 | mg | 0.88 ml | 1 | ml |
| 40 | 80 | mg | 0.80 ml | 0.8 | ml | 40 | mg | 1.00 ml | 1 | ml |

| | | | | | |
|---|---|---|---|---|---|
| 50 | 50 | mg | 0.50 ml | 0.8 | ml |
| 60 | 60 | mg | 0.60 ml | 0.8 | ml |
| 70 | 70 | mg | 0.70 ml | 0.8 | ml |
| 80 | 80 | mg | 0.80 ml | 0.8 | ml |

| | | | | |
|---|---|---|---|---|
| 90 | 90 | mg | 0.90 ml | 0.8 ml (×2) |
| 100 | 100 | mg | 1.00 ml | 0.8 ml (×2) |

The following examples are intended to illustrate, rather than limit, the disclosure.

### EXAMPLES

### Example 1. Hypotonia in a Subject with an HPP-Like Disease with No Loss-of-Function Mutations in ALPL

Patient A was diagnosed with HPP at 5 years and 9 months when she presented for evaluation of decreased bone mineral density associated with severe hypotonia. She was found to have low alkaline phosphatase level at 149 U/L (150-420), elevated PLP at 172.4 (20-125) and elevated urinary PEA level at 190 mg/dl (0-106). Skeletal imaging showed normal x-rays of the wrists but x-rays of the knees showed decreased bone mineral density, gracile bones, and abnormal tibial epiphyses. DXA scan showed low bone mineral density (Z-scores of −4.6 and −3.3 for the lumbar spine and the total body less head, respectively). She has not had any fractures. She had ALPL gene sequencing including deletion and duplication studies and no mutations in the ALPL gene were identified.

Prior to her diagnosis of HPP, Patient A had extensive molecular testing in an effort to identify the cause of her severe hypotonia which began in infancy. During her first year of life, evaluations for spinal muscular atrophy, muscle biopsies, a nerve and electromyography (EMG) study did not yield a specific explanation for her persistent hypotonia and muscle weakness. SMA gene testing revealed no disease causing mutations. The muscle biopsy revealed a chronic and ongoing myopathy noting features of fiber type disproportion. The EMG interpretation revealed a possible mild abnormality of neuromuscular transmission. MRI of the brain revealed volume loss/hypoplasia involving the inferior portion of cerebellar vermis with mild bilateral cerebellar volume loss.

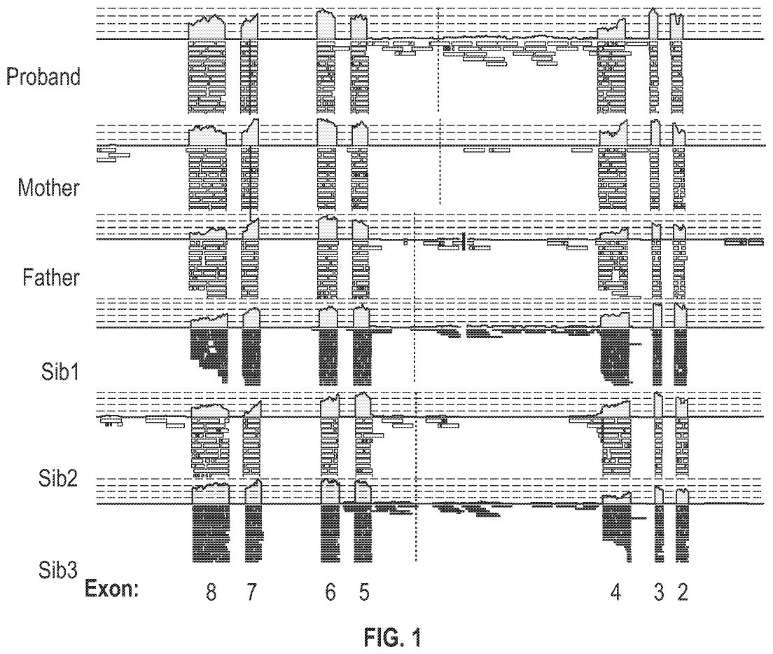

Subsequently, extensive genetic testing was performed, but did not yield a definitive explanation for Patient A's complex clinical picture. Comprehensive mitochondrial DNA (mtDNA) analysis panel was performed at Baylor College of Medicine and a rare variant of unknown significance was reported in the MTRNR2 gene (m.1836A>G). The Coenzyme Q10 Deficiency Panel was also performed at Baylor College of Medicine and a rare variant of unknown significance was reported in the ADCK3 gene (c.1665G>A). Whole exome sequencing (WES) was performed at GeneDx which revealed two variants, but neither explained her phenotype. One variant was the known ADCK3 variant previously identified by the Baylor Coenzyme Q10 Deficiency Panel. Although this variant is likely pathogenic, it is associated with autosomal recessive CoQ10 deficiency. This is not a significant explanation since Patient A only carries a maternal mutation. The second variant was a paternally inherited variant in the ATP1A3 gene. This variant (c.357+1G>A) is classified as likely pathogenic, but her father has the same variant and is unaffected. Therefore, the unaffected status of Patient A's father appears to be evidence that having the c.357+1G>A variant in the ATP1A3 gene is not causative of her clinical phenotype. Variant c.357+1G>A is located between exons 4 and 5 and may affect the splicing of these two exons. The mother does not carry this variant and exhibited a well-defined splice junction between exons 4 and 5 relative to other family members. Other family members showed varying degrees of “leak” between exons 4 and 5, with patient A exhibiting the largest numbers of reads mapped to the intron between exons 4 and 5, suggesting a less efficient splicing of the intron (FIG. 1). However, ATP1A3 is expressed at very low levels in PBMC and the reads mapped to the affected intron were low.

### Example 2. Quantifying ALPL Transcription and Translation

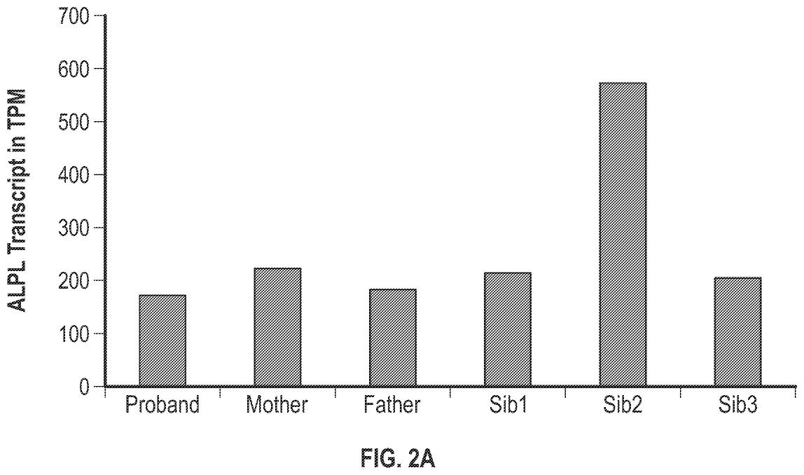

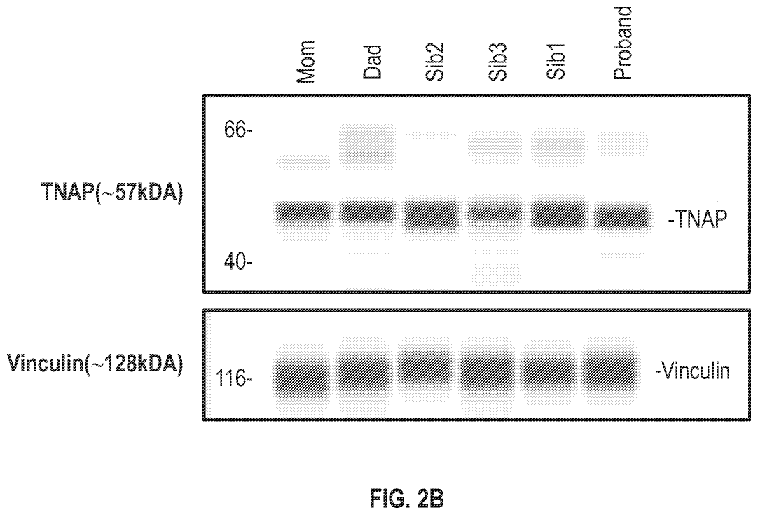

Patient A was subsequently examined for an impairment in ALPL gene expression due to ALPL transcription dysregulation at the RNA and/or protein level by RNA sequencing of ALPL transcript and Western analysis of ALPL protein, respectively. RNA sequencing showed that ALPL transcript level was normal in Patient A as well as in Patient A's mother, father, and three siblings (FIG. 2A). Western blot analysis was used to identify translation of ALPL. The analysis confirmed that TNAP protein was produced in Patient A as well as in Patient A's mother, father, and three siblings (FIG. 2B). Confirmatory analysis using a positive control and quantitative analysis will be performed.

### Example 3. Quantifying ALPL Translation

Western blot analysis of ALPL can be performed using a sample from Patient A to determine if the ALPL protein is produced at normal levels and at the correct molecular weight. A determination that the protein is not present at normal concentrations, even though patient A exhibits normal transcript levels, indicates a likely defect in ALPL translation.

Patient A can be subsequently analyzed along with her parents and three siblings in order to obtain a complete genetic profile. The study will include informed consent/assent, collection of a blood sample (20 mL) from Patient A, her parents, and her three siblings for Western blot analysis, RT-PCR analysis, or RNA Seq. of ALPL transcript and Western Blot analysis of ALPL protein. In addition, blood (7 mL) will be collected for a determination of PLP, Vitamin D2+D3, comprehensive metabolic panel, and urine PEA measurements from the parents and siblings. A medical history questionnaire and anthropometrics medical records review will also be conducted.

### Example 4. Treating Hypotonia with Asfotase Alfa

A 12 year old female with chromosomal duplication 22q11.21q11.22, developmental delay, autism spectrum disorder (Asperger's syndrome), extreme fatigue, pain that wakes her up at night (shoulders, back, legs) as well as findings consistent with HPP (low ALP, elevated PLP and PEA, and decreased bone mineralization) was initially diagnosed as having HPP. It was later discovered that the patient had no ALPL mutations. Within months of starting STRENSIQ®, the female patient's strength, agility, and endurance improved. Her 6MWT improved from 320 meters to 597 meters (age/gender norm is 672±55). Overall, she exhibited less pain (2 out of 10 instead of 5 out of 10 reported during previous visit) and improved mobility.

### Example 5. Treatments can Address Muscle Weakness, as Measured by Reduced SRC, that is Independent of Bone Mineralization Status

Experiments were performed to analyze the effects of fusion proteins on muscle weakness, as measured by reduced SRC, that is independent of bone mineralization status. For context, fusion proteins from two variants of SEQ ID NO: 1 were considered, including SEQ ID NO: 21, and SEQ ID NO: 22. A comparison of the three fusion protein structures is shown in Table 2, and the exact sequences are illustrated in Tables, 3, 4, and 5. Additional data for treatment with the fusion protein of SEQ ID NO: 21 is included, below.

TABLE 2
Fusion Protein Comparisons

| | SEQ ID NO: 1 | SEQ ID NO: 21 | SEQ ID NO: 22 |
|---|---|---|---|
| | (726 Amino Acids) | (724 Amino Acids) | (721 Amino Acids) |
| Catalytic domain | wild type | E108M/N213Q/N286Q | E108M |
| Fc region | Truncated IgG1 | Truncated IgG2/4 | Truncated IgG2/4 |
| Polyaspartate tag | D10 | D10 | D7 |

TABLE 3
SEQ ID NO: 1 (726 Amino Acids)

LVPEKEKDPKYWRDQAQETLKYALELQKLNTNVAKNVIMFLGDGMGVSTVTAARILKGQLHHN
PGEETRLEMDKFPFVALSKTYNTNAQVPDSAGTATAYLCGVKANEGTVGVSAATERSRCNTTQG
NEVTSILRWAKDAGKSVGIVTTTRVNHATPSAAYAHSADRDWYSDNEMPPEALSQGCKDIAYQL
MHNIRDIDVIMGGGRKYMYPKNKTDVEYESDEKARGTRLDGLDLVDTWKSFKPRYKHSHFIWN
RTELLTLDPHNVDYLLGLFEPGDMQYELNRNNVTDPSLSEMVVVAIQILRKNPKGFFLLVEGGRID
HGHHEGKAKQALHEAVEMDRAIGQAGSLTSSEDTLTVVTADHSHVFTFGGYTPRGNSIFGLAPM
LSDTDKKPFTAILYGNGPGYKVVGGERENVSMVDYAHNNYQAQSAVPLRHETHGGEDVAVFSK
GPMAHLLHGVHEQNYVPHVMAYAACIGANLGHCAPASSLKDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVFSCSVMHEALHNHYTQ
KSLSLSPGKDIDDDDDDDDDD

TABLE 4
SEQ ID NO: 21 (724 AA) E108M, N213Q, N286Q

LVPEKEKDPKYWRDQAQETLKYALELQKLNTNVAKNVIMFLGDGMGVSTVTAARILKGQL
HHNPGEETRLEMDKFPFVALSKTYNTNAQVPDSAGTATAYLCGVKANMGTVGVSAATERS
RCNTTQGNEVTSILRWAKDAGKSVGIVTTTRVNHATPSAAYAHSADRDWYSDNEMPPEAL
SQGCKDIAYQLMHNIRDIDVIMGGGRKYMYPKQKTDVEYESDEKARGTRLDGLDLVDTWK
SFKPRYKHSHFIWNRTELLTLDPHNVDYLLGLFEPGDMQYELNRNQVTDPSLSEMVVVAI
QILRKNPKGFFLLVEGGRIDHGHHEGKAKQALHEAVEMDRAIGQAGSLTSSEDTLTVVTA
DHSHVFTFGGYTPRGNSIFGLAPMLSDTDKKPFTAILYGNGPGYKVVGGERENVSMVDYA
HNNYQAQSAVPLRHETHGGEDVAVFSKGPMAHLLHGVHEQNYVPHVMAYAACIGANLGHC
APASSAGSLAAVECPPCPAPPVAGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKDDDDDD
DDDD

TABLE 5
SEQ ID NO: 22 (721 AA) E108M, IgG2/4, D7

LVPEKEKDPKYWRDQAQETLKYALELQKLNTNVAKNVIMFLGDGMGVSTVTAARILKGQL
HHNPGEETRLEMDKFPFVALSKTYNTNAQVPDSAGTATAYLCGVKANMGTVGVSAATERS
RCNTTQGNEVTSILRWAKDAGKSVGIVTTTRVNHATPSAAYAHSADRDWYSDNEMPPEAL
SQGCKDIAYQLMHNIRDIDVIMGGGRKYMYPKNKTDVEYESDEKARGTRLDGLDLVDTWK
SFKPRYKHSHFIWNRTELLTLDPHNVDYLLGLFEPGDMQYELNRNNVTDPSLSEMVVVAI
QILRKNPKGFFLLVEGGRIDHGHHEGKAKQALHEAVEMDRAIGQAGSLTSSEDTLTVVTA
DHSHVFTFGGYTPRGNSIFGLAPMLSDTDKKPFTAILYGNGPGYKVVGGERENVSMVDYA
HNNYQAQSAVPLRHETHGGEDVAVFSKGPMAHLLHGVHEQNYVPHVMAYAACIGANLGHC
APASSAGSLAAVECPPCPAPPVAGPSVFLFPPKPKDTLMISRTPEVTCVVVDVSQEDPEV
QFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLSLSLGKDDDDDDD

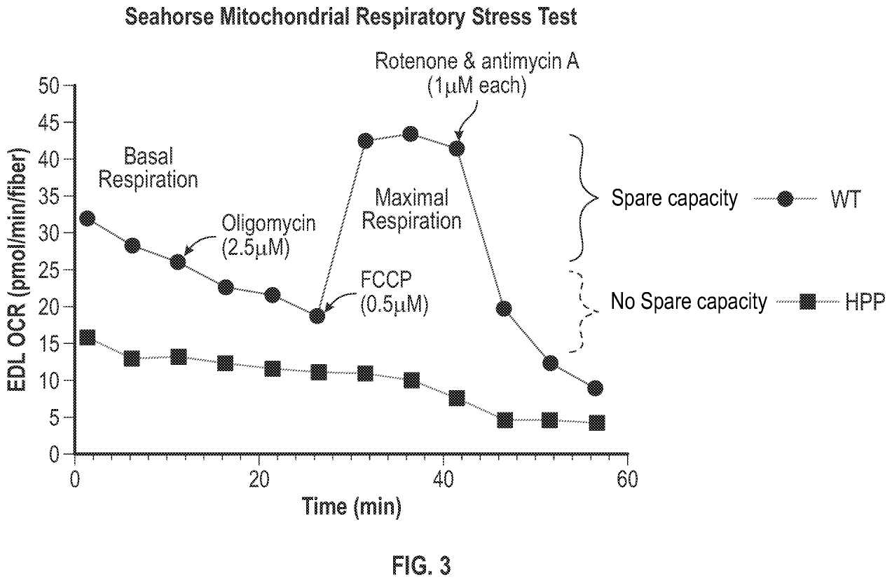

FIG. 3 depicts the representative oxygen consumption rate (OCR) profile normalized to extensor digitorum longus (EDL) skeletal muscle fiber number from a postnatal day 20 female HPP mouse and WT female littermate, demonstrating that HPP mouse EDL muscle fibers can be deficient in spare respiratory capacity compared to age and sex matched WT. These data suggest that reduced mitochondrial energy production rates contribute to muscle weakness in HPP. Abbreviations of FIG. 3 are: ATP=adenosine triphosphate; EDL=extensor digitorum longus; FCCP=carbonyl cyanide-ptrifluoromethoxy phenylhydrazone; HPP=hypophosphatasia; OCR=oxygen consumption rate; WT=wild type.

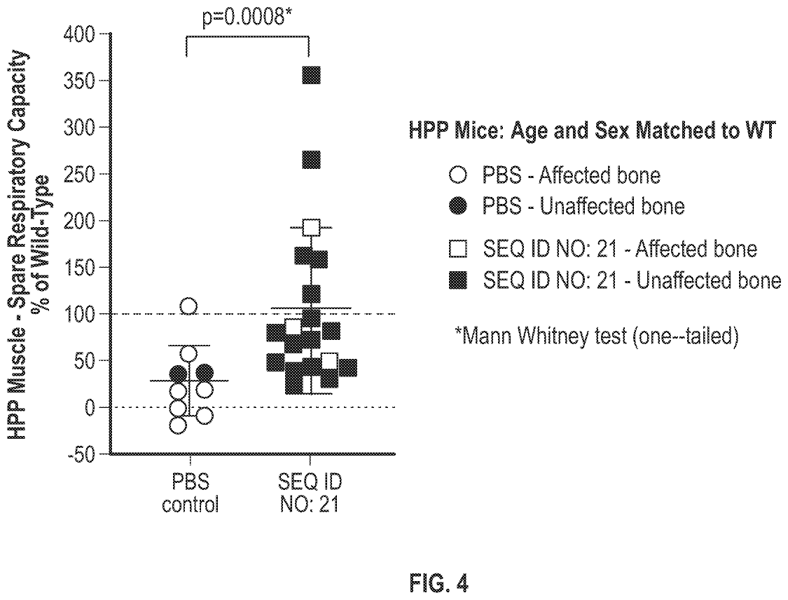

Spare respiratory capacity (SRC) expressed as percent of WT was comparatively evaluated in PBS-treated and HPP mice treated with the fusion protein for SEQ ID NO: 21 (FIG. 4 and Table 6). Table 6 abbreviations are: Abbreviations: HPP=hypophosphatasia; PBS=phosphate buffer saline; SRC=spare respiratory capacity; WT=wildtype.

TABLE 6
Spare respiratory capacity (SRC) expressed as percent of WT

| | HPP muscle SRC (percent of | |
|---|---|---|
| | WT) | HPP bone phenotype |
| Treatment | Mean ± SD | Percent Unaffected |
| PBS | 29.65 ± 37.44 | 22 |
| SEQ ID NO: 21 | 107.48 ± 87.36 | 84 |

The SRC of HPP mice treated with the fusion protein of SEQ ID NO: 21, expressed as percent of WT, was significantly increased compared to HPP mice treated with PBS alone (p=0.0008 using a one-tailed Mann Whitney test). These data demonstrate the ability of the treatment with the fusion protein of SEQ ID NO: 21 to improve mitochondrial bioenergetics in skeletal muscle fibers of HPP mice. Bone phenotyping of PBS control mice revealed unaffected bone in 2 of 9 HPP mice (22%), despite these mice having less than 50% of the SRC of matched WT. HPP mice can have muscle weakness, as measured by reduced SRC, that is independent of bone mineralization status. These data suggest that patients with HPP who suffer from muscle weakness without skeletal manifestations could benefit from the treatment with fusion protein of SEQ ID NO: 21. Treatment with fusion protein of SEQ ID NO: 21 rescued the bone phenotype in all but 3 of 19 HPP mice (84%). These 3 mice received 8, 9 or 13 doses, which is fewer than the 18 doses of 2 mg/kg of the fusion protein of SEQ ID NO: 21 that normalized bone mineralization phenotype in 94% of HPP mice.

From Example 5 data, one finds support for a method of treating or reducing the risk of developing muscle weakness in a subject having or at risk of having a muscle weakness disease comprising administering to the subject a therapeutically effective amount of at least one recombinant polypeptide having alkaline phosphatase activity, wherein the subject exhibits a reduced concentration of tissue non-specific alkaline phosphatase (TNSALP), and wherein the subject does not have a loss-of-function mutation in an ALPL gene. In specific embodiments, the method comprises a fusion protein comprising an amino acid sequence comprising at least 95% sequence identity to SEQ ID NO: 21 or SQ ID NO: 22 (or wherein the recombinant polypeptide comprises a fusion protein comprising an amino acid of SEQ ID NO: 21 or SQ ID NO: 22). In specific embodiments, the recombinant polypeptide of the method comprises an immunoglobulin molecule, the immunoglobulin molecule is An IgG2/4 fragment crystallizable (Fc) region, and the Fc comprises an amino acid sequence of SEQ ID NO: 21. The administration of the therapeutically effective amount in specific embodiments is biweekly, and can comprise from about 0.1 mg/kg to about 20 mg/kg. In specific embodiments, the method of claim 66, wherein muscle weakness disease comprises: muscular dystrophy, myasthenia gravis, and calcium pyrophosphate deposition disease (CPPD), amyotrophic lateral sclerosis (ALS), myositis, myotonic dystrophy, myotonia, Guillain-Barre syndrome, Duchenne muscular dystrophy (DMD), or Lambert-Eaton myasthenic syndrome. The administration of the method can improve mitochondrial bioenergetics in skeletal muscle fibers.

### Example 6. SEQ ID NO: 1 Restores Pyridoxal Phosphate (PLP)-Dependent BCAT Activity to Enable Adenosine Triphosphate Production from Branched Chain Amino Acid Transferases

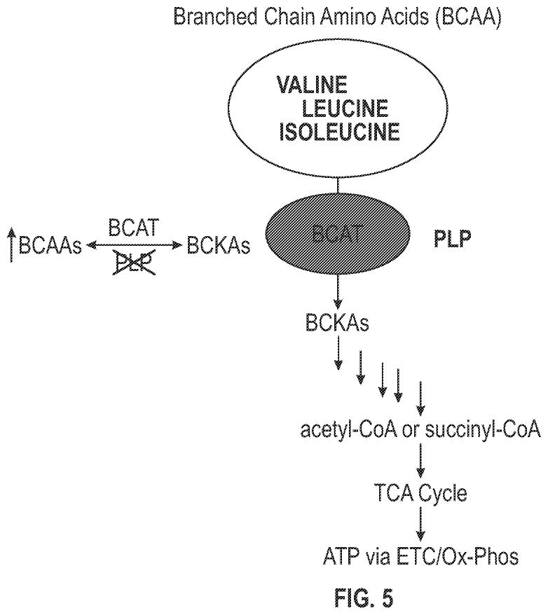

In the absence of continuous SEQ ID NO: 1 treatment, branched chain amino acids (BCAAs) (valine, leucine, and isoleucine) are elevated in the skeletal muscle of HPP mice (TNSALP knockout) due to insufficient intracellular pyridoxal phosphate (PLP) for PLP-dependent branched chain amino acid transferase (BCAT) catabolism of BCAAs to branched chain keto acids (BCKAs). (BCKAs are catabolized (series of sequential steps) to generate acetyl-CoA or succinyl-CoA that can enter the mitochondrial tricarboxylic acid (TCA) cycle to produce adenosine triphosphate (ATP) via oxidative phosphorylation of adenosine diphosphate (ADP)). The pathway is shown in FIG. 5.

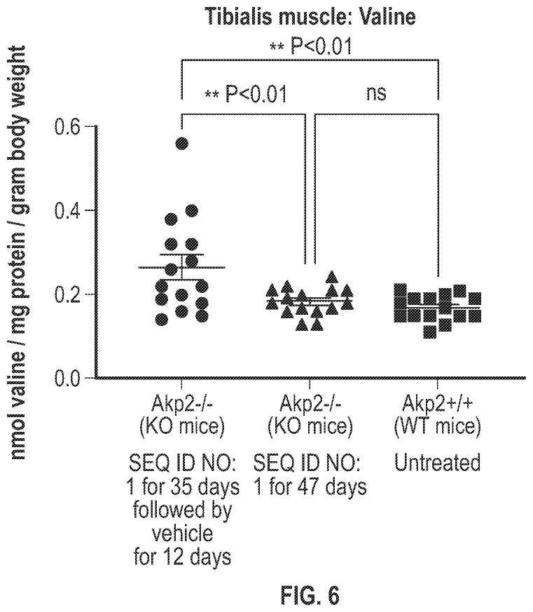

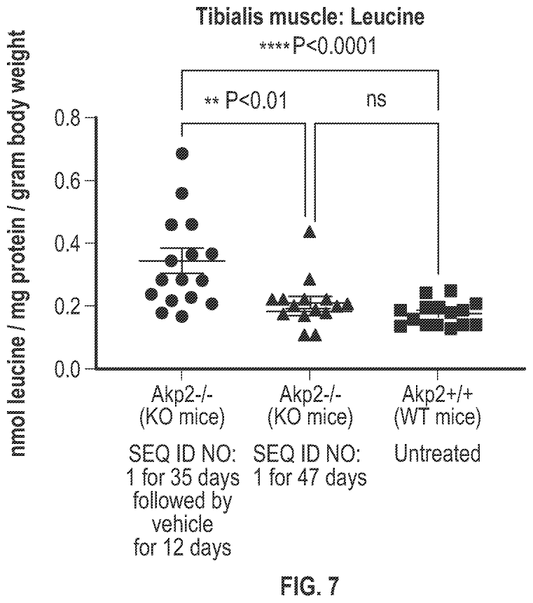

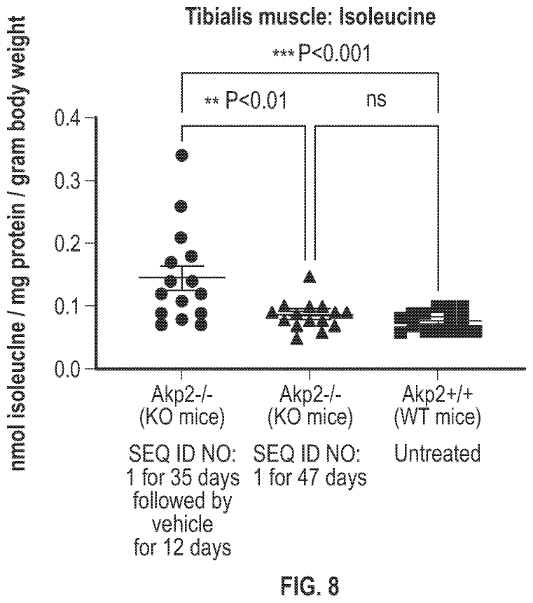

SEQ ID NO: 1 treatment of HPP mice restores PLP-dependent BCAT activity to normalize BCAAs in skeletal muscle (makes BCAAs available for energy production). This is shown in FIGS. 6, 7, and 8 where continuous SEQ ID NO: 1 treatment normalizes BCAA levels in HPP mice skeletal muscle (no significant difference compared to wild-type), providing indirect evidence that PLP-dependent BCAT activity is restored, enabling ATP production from BCAAs. Discontinuation (withdrawal) of SEQ ID NO: 1 treatment in TNSALP knockout mice significantly increases the levels of valine, leucine, and isoleucine in the tibialis muscle compared to wild-type mice, providing indirect evidence that PLP-dependent BCAT activity is reduced, thereby limiting ATP production from BCAAs.

### OTHER EMBODIMENTS

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits will be apparent to those skilled in the art without departing from the scope and spirit of the claimed invention. Although the disclosure has been described in connection with specific embodiments, it will be understood that it is capable of further modifications and that the invention as claimed should not be unduly limited to such specific embodiments.

1. A method of treating or reducing the risk of developing muscle weakness in a subject having or at risk of having a muscle weakness disease comprising administering to the subject a therapeutically effective amount of at least one recombinant polypeptide having alkaline phosphatase activity, wherein the subject exhibits a reduced concentration of tissue non-specific alkaline phosphatase (TNSALP), and wherein the subject does not have a loss-of-function mutation in an ALPL gene.

2. The method of claim 1, wherein the reduced concentration of TNSALP is caused by reduced transcription of ALPL, reduced translation of ALPL mRNA, elevated or reduced posttranslational modification of TNSALP, or reduced of TNSALP enzymatic activity.

3. The method of claim 1 or 2, wherein the reduced transcription of ALPL, reduced translation of ALPL mRNA, elevated or reduced posttranslational modification of TNSALP, or reduced TNSALP enzymatic activity is measured relative to a normal subject.

4. The method of any one of claims 1 to 3, wherein the subject has a mutation in a 3′ untranslated region (UTR), a 5′ UTR, or an intronic region of the ALPL gene.

5. The method of any one of claims 1 to 4, wherein the subject has a mutation in one or more of ATP1A3, ANKH, ENPP1, FGFR3, PHOSPHO1, PTHIR, PTH2R, SPP1, TNFRSF11A, TNFRSF11B, COL1A1, COL1A2, SOX9, PDXP, AOX1, PNPO, PDXK, ADCK3, MTRNR2, and S1PR1.

6. The method of claim 5, wherein the subject has a mutation in ATP1A3.

7. The method of claim 5 or 6, wherein the subject has a mutation in ADCK3.

8. The method of any one of claims 1 to 7, wherein a muscle of said subject is not significantly different from a muscle of a normal subject without said muscle weakness disease in at least one property selected from muscle fiber type proportion and fiber contractile properties.

9. The method of any one of claims 1 to 8, wherein the muscle is at least one type of leg muscle, wherein, optionally, the at least one type of muscle is selected from a soleus muscle and an extensor digitorum longus (EDL) muscle.

10. The method of any one of claims 1 to 9, wherein the muscle weakness disease is caused by reduced alkaline phosphatase activity.

11. The method of any one of claims 1 to 10, wherein the subject has an elevated serum concentration of pyrophosphate (PPi), wherein, optionally, the muscle weakness disease is caused by the elevated concentration of PPi.

12. The method of claim 11, wherein the elevated concentration of pyrophosphate (PPi) enhances muscle weakness in said subject.

13. The method of any one of claims 1 to 12, wherein the recombinant polypeptide reduces the concentration of PPi in the subject.

14. The method of any one of claims 1 to 13, wherein the recombinant polypeptide is administered to the subject daily for at least one week, one month, three months, six months, or one year, or more.

15. The method of any one of claims 1 to 14, wherein the recombinant polypeptide is administered subcutaneously, intravenously, intramuscularly, sublingually, intrathecally, or intradermally.

16. The method of claim 15, wherein the recombinant polypeptide is administered subcutaneously.

17. The method of any one of claims 1 to 16, wherein the recombinant polypeptide comprises at least one of a tissue nonspecific alkaline phosphatase (TNALP), a placental alkaline phosphatase (PALP), a germ cell alkaline phosphatase (GCALP), an intestinal alkaline phosphatase (IALP), and biologically functional fragments, fusions, or chimeric constructs thereof.

18. The method of claim 17, wherein the recombinant polypeptide comprises at least one of a soluble fragment of TNALP, PALP, GCALP, and IALP.

19. The method of claim 17 or 18, wherein the tissue nonspecific alkaline phosphatase (TNALP) comprises an amino acid sequence of the amino acids 1-485 of SEQ ID NO: 1.

20. The method of any one of claims 1 to 19, wherein the recombinant polypeptide is a fusion protein.

21. The method of any one of claims 1 to 20, wherein the recombinant polypeptide comprise an immunoglobulin molecule.

22. The method of claim 21, wherein the immunoglobulin molecule is a fragment crystallizable (Fc) region.

23. The method of claim 22, wherein the Fc comprises an amino acid sequence of SEQ ID NO: 20.

24. The method of any one of claims 1 to 23, wherein the recombinant polypeptide comprises a negatively charged peptide.

25. The method of claim 24, wherein the negatively charged peptide comprises one to fifty negatively charged amino acids, such as an aspartic acid or glutamic acid, wherein, optionally, the negatively charged peptide is at least one of D₁₀, D₁₆, E₁₀, and E₁₆.

26. The method of any one of claims 1 to 18, wherein the recombinant polypeptide comprises a bone targeted alkaline phosphatase having the structure:
Z-sALP-Y-spacer-X-Wₙ-V,
wherein sALP is the extracellular domain of the alkaline phosphatase;
V is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid; and
Wₙ is a polyaspartate or a polyglutamate wherein n=10 to 16.

27. The method of claim 26, wherein the spacer comprises a fragment crystallizable region (Fc).

28. The method of claim 27, wherein the Fc comprises an amino acid sequence of SEQ ID NO: 20.

29. The method of claim 27 or 28, wherein the recombinant polypeptide comprises the structure of SALP-Fc-D₁₀.

30. The method of any one of claims 1 to 29, wherein the recombinant polypeptide is administered at a dosage of from about 0.1 mg/kg/day to about 20 mg/kg/day, or a comparable weekly dosage.

31. The method of claim 30, wherein the recombinant polypeptide is administered at a dosage of from about 0.5 mg/kg/day to about 20 mg/kg/day, or a comparable weekly dosage.

32. The method of claim 31, wherein the recombinant polypeptide is administered at a dosage of from about 0.5 mg/kg/day to about 10 mg/kg/day, or a comparable weekly dosage.

33. The method of claim 32, wherein the recombinant polypeptide is administered at a dosage of from about 1 mg/kg/day to about 10 mg/kg/day, or a comparable weekly dosage.

34. The method of claim 33, wherein the recombinant polypeptide is administered at a dosage of about 6 mg/kg/week.

35. The method of claim 34, wherein the recombinant polypeptide is administered at a dosage of about 1 mg/kg 6 times per week, about 2 mg/kg 3 times per week, or 3 mg/kg 2 times per week.

36. The method of any one of claims 1 to 35, wherein, prior to administration of the recombinant polypeptide, the subject is characterized as having an average walking distance in six minutes of about 350 meters or less.

37. The method of any one of claims 1 to 36, wherein administration of the recombinant polypeptide promotes an increase in an average walking distance in six minutes by the subject of at least 100 meters or more.

38. The method of any one of claims 1 to 37, wherein the subject exhibits an average walking distance in six minutes of about 500 meters or more after administration of the recombinant polypeptide.

39. The method of any one of claims 1 to 38, wherein the subject exhibits decreased reliance on an assistive mobility device after administration of the recombinant polypeptide.

40. The method of claim 39, wherein the assistive mobility device is at least one device selected from the group consisting of a walker, a wheelchair, braces, crutches, and orthotics.

41. The method of any one of claims 1 to 40, wherein, prior to administration of the recombinant polypeptide, the subject is characterized as having a plasma PPi concentration of about 4.5 μM or greater.

42. The method of any one of claims 1 to 41, wherein administration of the recombinant polypeptide promotes a median decrease in PPi concentration in a plasma sample from the subject of at least about 1 M.

43. The method of any one of claims 1 to 42, wherein the subject exhibits a plasma PPi concentration of about 2 μM to about 5 UM after administration of the recombinant polypeptide.

44. The method of any one of claims 1 to 43, wherein:
i) the subject is 0 to 14 days of age and, prior to administration of the recombinant polypeptide, is characterized as having a plasma ALP concentration of about 90 U/L or less;
ii) the subject is 15 days to less than 1 year of age and, prior to administration of the recombinant polypeptide, is characterized as having a plasma ALP concentration of about 134 U/L or less;
iii) the subject is about 1 year to less than 10 years of age and, prior to administration of the recombinant polypeptide, is characterized as having a plasma ALP concentration of about 156 U/L or less;
iv) the subject is about 10 years to about 13 years of age and, prior to administration of the recombinant polypeptide, is characterized as having a plasma ALP concentration of about 141 U/L or less;
v) the subject is female and about 13 years to about 15 years of age and, prior to administration of the recombinant polypeptide, is characterized as having a plasma ALP concentration of about 62 U/L or less;
vi) the subject is male and about 13 years to about 15 years of age and, prior to administration of the recombinant polypeptide, is characterized as having a plasma ALP concentration of about 127 U/L or less;
vii) the subject is female and about 15 years to about 17 years of age and, prior to administration of the recombinant polypeptide, is characterized as having a plasma ALP concentration of about 54 U/L or less;
viii) the subject is male and about 15 years to about 17 years of age and, prior to administration of the recombinant polypeptide, is characterized as having a plasma ALP concentration of about 89 U/L or less;
ix) the subject is about 17 years of age or older and, prior to administration of the recombinant polypeptide, is characterized as having a plasma ALP concentration of about 48 U/L or less; or
x) the subject is about 17 years of age or older and, prior to administration of the recombinant polypeptide, is characterized as having a plasma ALP concentration of about 59 U/L or less.

45. The method of any one of claims 1 to 44, wherein administration of the recombinant polypeptide promotes a median increase in ALP concentration in a plasma sample from the subject of at least about 100 U/L or greater.

46. The method of any one of claims 1 to 45, wherein:
i) the subject is 0 to 14 days of age and, after administration of the recombinant polypeptide, is characterized as having a plasma ALP concentration of about 273 U/L or greater;
ii) the subject is 15 days to less than 1 year of age and, after administration of the recombinant polypeptide, is characterized as having a plasma ALP concentration of about 518 U/L or greater;
iii) the subject is about 1 year to less than about 10 years of age and, after administration of the recombinant polypeptide, is characterized as having a plasma ALP concentration of about 369 U/L or greater;
iv) the subject is about 10 years to about 13 years of age and, after administration of the recombinant polypeptide, is characterized as having a plasma ALP concentration of about 460 U/L or greater;
v) the subject is female and about 13 years to about 15 years of age and, after administration of the recombinant polypeptide, is characterized as having a plasma ALP concentration of about 280 U/L or greater;
vi) the subject is male and about 13 years to about 15 years of age and, after administration of the recombinant polypeptide, is characterized as having a plasma ALP concentration of about 517 U/L or greater;
vii) the subject is female and about 15 years to about 17 years of age and, after administration of the recombinant polypeptide, is characterized as having a plasma ALP concentration of about 128 U/L or greater;
viii) the subject is male and about 15 years to about 17 years of age and, after administration of the recombinant polypeptide, is characterized as having a plasma ALP concentration of about 365 U/L or greater;
ix) the subject is female and about 17 years of age or older and, after administration of the recombinant polypeptide, is characterized as having a plasma ALP concentration of about 95 U/L or greater; or
x) the subject is male and about 17 years of age or older and, after administration of the recombinant polypeptide, is characterized as having a plasma ALP concentration of about 164 U/L or greater.

47. The method of any one of claims 1 to 46, wherein, prior to administration of the recombinant polypeptide, the subject is characterized as having an average Bruininks-Oseretsky Test of Motor Proficiency 2nd Edition (BOT-2) strength score of about 10 or less.

48. The method of claim 47, wherein, prior to administration of the recombinant polypeptide, the subject is characterized as having an average BOT-2 running speed and agility score of about 5 or less.

49. The method of any one of claims 1 to 48, wherein administration of the recombinant polypeptide results in an average BOT-2 strength score of the subject of about 10 or more.

50. The method of any one of claims 1 to 49, wherein administration of the recombinant polypeptide results in an average BOT-2 running speed and agility score of the subject of about 5 or more.

51. The method of any one of claims 1 to 50, wherein, prior to administration of the recombinant polypeptide, the subject is characterized as having an average Childhood Health Assessment Questionnaire (CHAQ) index score of about 0.8 or more.

52. The method of any one of claims 1 to 51, wherein administration of the recombinant polypeptide results in an average CHAQ index score of the subject of about 0.5 or less.

53. The method of any one of claims 1 to 52, wherein, prior to administration of the recombinant polypeptide, the subject is characterized as having an average Pediatric Outcomes Data Collection Instrument (PODCI) score of about 40 or less.

54. The method of any one of claims 1 to 53, wherein administration of the recombinant polypeptide results in an average PODCI score of the subject of about 40 or more.

55. The method of any one of claims 1 to 54, wherein, prior to administration of the recombinant polypeptide, the subject is characterized as having an average Muscle Strength Grade of less than about 5.

56. The method of any one of claims 1 to 55, wherein administration of the recombinant polypeptide results in an average increase in a Muscle Strength Grade of the subject of about 1 or more.

57. The method of any one of claims 1 to 56, wherein, prior to administration of the recombinant polypeptide, the subject is characterized as having an average Hand Held Dynamometry (HHD) value of less than about 80% of a predicted HHD value.

58. The method of any one of claims 1 to 57, wherein administration of the recombinant polypeptide results in an average HHD value of the subject of about 80% or more of a predicted HHD value.

59. The method of claim 57 or 58, wherein the HHD value represents the grip strength, knee flexion, knee extension, hip flexion, hip extension, or hip abduction of the subject.

60. The method of any one of claims 1 to 59, wherein the subject has a muscle weakness disease selected from muscular dystrophy, myasthenia gravis, and calcium pyrophosphate deposition disease (CPPD), amyotrophic lateral sclerosis (ALS), myositis, myotonic dystrophy, myotonia, Guillain-Barre syndrome, Duchenne muscular dystrophy (DMD), and Lambert-Eaton myasthenic syndrome.

61. The method of claim 1, wherein the recombinant polypeptide comprises a fusion protein comprising an amino acid comprising at least 95% sequence identity to SEQ ID NO: 21 or SEQ ID NO: 22.

62. The method of claim 61, wherein the recombinant polypeptide comprises a fusion protein comprising an amino acid sequence of SEQ ID NO: 21 or SEQ ID NO: 22.

63. The method of claim 1, wherein: the recombinant polypeptide comprises an immunoglobulin molecule, the immunoglobulin molecule is An IgG2/4 fragment crystallizable (Fc) region, and the Fc comprises an amino acid sequence of SEQ ID NO: 21.

64. The method of claim 61, wherein the administration of the therapeutically effective amount is biweekly.

65. The method of claim 61, wherein the administration of the therapeutically effective amount is from about 0.1 mg/kg to about 20 mg/kg.

66. The method of claim 65, wherein muscle weakness disease comprises: muscular dystrophy, myasthenia gravis, and calcium pyrophosphate deposition disease (CPPD), amyotrophic lateral sclerosis (ALS), myositis, myotonic dystrophy, myotonia, Guillain-Barre syndrome, Duchenne muscular dystrophy (DMD), or Lambert-Eaton myasthenic syndrome.

67. The method of claim 61, wherein the administration improves mitochondrial bioenergetics in skeletal muscle fibers.

68. The method of claim 1, wherein the treatment with the therapeutically effective amount of the at least one recombinant polypeptide having the alkaline phosphatase activity results in a decrease in a level of valine, leucine, or isoleucine in a muscle of the subject.


SEQUENCE LISTING (22 sequences)

SEQ ID NO: 1 - protein, 726 aa, synthetic construct
  REGION 1..726: note = Synthetic Construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 2 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 3 - protein, 524 aa, Homo sapiens [sequence omitted]

SEQ ID NO: 4 - protein, 524 aa, Homo sapiens; identical to SEQ ID NO: 2

SEQ ID NO: 5 - protein, 524 aa, Homo sapiens; identical to SEQ ID NO: 2

SEQ ID NO: 6 - protein, 524 aa, Homo sapiens [sequence omitted]

SEQ ID NO: 7 - protein, 652 aa, Macaca mulatta
MPTVKTKQESHAGSGSGPRLAERKGRVGAARRQSPRAPGGGLPGPRSGPAAAFIRRRGRWPGPRCAPATPRPRSRLCAPTRLCLDEPSSVLCAGLEHQLTSDHCQPTPSHPRRSHLWASGIKQVLGCTMISPFLVLAIGTCLTNSLVPEKEKDPKYWRDQAQETLKYALELQKLNTNVAKNVIMFLGDGMGVSTVTATRILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLVHNIRDIDVIMGGGRKYMYPKNKTDVEYEIDEKARGTRLDGLDLVNIWKSFKPRHKHSHFIWNRTELLTLDPHNVDYLLGLFEPGDMEYELNRNNVTDPSLSEMVVVAIQILRKNPKGFFLLVEGGRIDHGHHEGKAKQALHEAVEMDRAIGQAGSMTSLEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYIPHVMAYAACIGANLDHCAPASSAGSLAAGPLLLPLALFPLSILF

SEQ ID NO: 8 - protein, 524 aa, Rattus norvegicus
MILPFLVLAIGPCLTNSFVPEKEKDPSYWRQQAQETLKNALKLQKLNTNVAKNIIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMRPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNPKGFFLLVEGGRIDHGHHEGKAKQALHEAVEMDEAIGKAGTMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWASSASSPSPGALLLPLALFPLRTLF

SEQ ID NO: 9 - protein, 502 aa, Canis lupus
  source 1..502: note = Canis lupus familiaris
EKDPKYWRDQAQQTLKYALRLQNLNTNVAKNVIMFLGDGMGVSTVTATRILKGQLHHNPGEETRLEMDKFPYVALSKTYNTNAQVPDSAGTATAYLCGVKANEGTVGVSAATQRTHCNTTQGNEVTSILRWAKDAGKSVGIVTTTRVNHATPSAAYAHSADRDWYSDNEMPPEALSQGCKDIAYQLMHNVKDIEVIMGGGRKYMFPKNRTDVEYEMDEKSTGARLDGLNLIDIWKNFKPRHKHSHYVWNRTELLALDPYTVDYLLGLFDPGDMQYELNRNNVTDPSLSEMVEIAIKILSKKPRGFFLLVEGGRIDHGHHEGKAKQALHEAVEMDRAIGKAGVMTSLEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANQDHCASASSAGGPSPGPLLLLLALLPVGILF

SEQ ID NO: 10 - protein, 252 aa, Sus scrofa
AELLALDPHTVDYLLGLFEPGDMQYELNRNNVTDPSLSEMVEMAIRILIKNPKGFFLLVEGGRIDHGHHEGKAKQALHEAVEMDRAIEQAGSMTSVEDTLTVVTADHSHVFTFGGYTPRGNSIFGLAPMVSDTDKKPFTAILYGNGPGYKVVGGERENVSMVDYAHDNYQAQSAVPLRHETHGGEDVAIFARGPMAHLLHGVHEQNYIPHVMAYAACVGANRDHCASASSSGSPSPGPLLLLLALLPLGILF

SEQ ID NO: 11 - protein, 524 aa, Mus musculus
MISPFLVLAIGTCLTNSFVPEKERDPSYWRQQAQETLKNALKLQKLNTNVAKNVIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMPPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNLKGFFLLVEGGRIDHGHHEGKAKQALHEAVEMDQAIGKAGAMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWAGSGSAPSPGALLLPLAVLSLRTLF

SEQ ID NO: 12 - protein, 524 aa, Bos taurus
MISPFLLLAIGTCFASSLVPEKEKDPKYWRDQAQQTLKNALRLQTLNTNVAKNVIMFLGDGMGVSTVTAARILKGQLHHSPGEETKLEMDKFPYVALSKTYNTNAQVPDSAGTATAYLCGVKANEGTVGVSAATQRSQCNTTQGNEVTSILRWAKDAGKSVGIVTTTRVNHATPSASYAHSADRDWYSDNEMPPEALSQGCKDIAYQLMHNIKDIEVIMGGGRKYMFPKNRTDVEYELDEKARGTRLDGLNLIDIWKSFKPKHKHSHYVWNRTDLLALDPHSVDYLLGLFEPGDMQYELNRNNATDPSLSEMVEMAIRILNKNPKGFFLLVEGGRIDHGHHEGKAKQALHEAVEMDQAIGQAGAMTSVEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANRDHCASASSSGSPSPGPLLLLLALLPLGSLF

SEQ ID NO: 13 - protein, 524 aa, Bos taurus; identical to SEQ ID NO: 12

SEQ ID NO: 14 - protein, 124 aa, Bos taurus
DPKYWRDQAQQTLKNALGLQKLNTKVAKNVILFLGDGMGVSTVTAARILKGQLHHNPGEETRLEMDKFPFVALSKTYNTNAQVPDSAGTAPHPVRVKAMRAPWGEPHQRQCNTRRATSTHLLAG

SEQ ID NO: 15 - protein, 524 aa, Felis catus
MISPFLVLAIGTCLTNSLVPEKEKDPKYWRDQAQQTLKNALRLQKLNTNVVKNVIMFLGDGMGVSTVTAARILKGQLHHNPGEETRLEMDKFPYVALSKTYNTNAQVPDSAGTATAYLCGVKANEGTVGVSAATQRTQCNTTQGNEVTSILRWAKDSGKSVGIVTTTRVNHATPSAAYAHSADRDWYSDNEMPPEALSQGCKDIAYQLMHNVRDIEVIMGGGRKYMFPKNRTDVEYEMDEKARGTRLDGLNLVDIWKSFKPRHKHSHYVWNRTELLTLDPYGVDYLLGLFEPGDMQYELNRNSTTDPSLSEMVEIAIKILSKNPKGFFLLVEGGRIDHGHHEGKAKQALHEAVEMDQAIGRAGAMTSVEDTLTIVTADHSHVFTFGGYTPRGNSIFGLAPMVSDTDKKPFTSILYGNGPGYKVVGGERENVSMVDYAHNNYQAQSAVPLRHETHGGEDVAVFAKGPMAHLLHGVHEQNYIPHVMAYAACIGANLDHCASASSAGGPSPGPLFLLLALPSLGILF

SEQ ID NO: 16 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 17 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 18 - protein, 532 aa, Homo sapiens; identical to SEQ ID NO: 16

SEQ ID NO: 19 - protein, 528 aa, Homo sapiens
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 20 - protein, 227 aa, synthetic construct
  REGION 1..227: note = Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 724 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANMGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKQKTDVEYESDEKARGTRLDGLDLVDTWKSFKPRYKHSHFIWNRTELLTLDPHNVDYLLGLFEPGDMQYELNRNQVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDDDDDDDDDD

SEQ ID NO: 22 - protein, 721 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANMGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDDDDDDD